(19) United States
(12) Patent Application Publication
(10) Pub. No.: US 2015/0065421 A1
(43) Pub. Date: Mar. 5, 2015

(54) BASAL INSULIN THERAPY

(71) Applicant: SANOFI, Paris (FR)

(72) Inventors: Peter Johnston, Bridgewater, NJ (US); Hertzel Gerstein, Hamilton (CA)

(21) Appl. No.: 14/387,791

(22) PCT Filed: Mar. 28, 2013
(86) PCT No.: PCT/EP2013/056661

§ 371 Date: Sep. 24, 2014

Related U.S. Application Data
(60) Provisional application No. 61/616,768, filed on Mar. 28, 2012.
(60) Provisional application No. 61/619,641, filed on Apr. 3, 2012.
(60) Provisional application No. 61/624,598, filed on Apr. 16, 2012.
(60) Provisional application No. 61/645,909, filed on May 11, 2012.
(60) Provisional application No. 61/701,911, filed on Sep. 17, 2012.

(51) Int. Cl. A61K 38/28; A61J 1/14

(52) U.S. Cl. CPC A61K 38/28; A61J 1/14
USPC 514/5.9; 206/232; 206/363

(57) ABSTRACT

The present invention relates to the use of use of a long-acting insulin, in particular insulin glargine, in a method of reducing the risk of progression to type 2 diabetes in a patient, a method of reducing the risk of a new angina in a patient and a method of reducing the risk of a microvascular event in a patient comprising administering to said patient in need thereof a therapeutically effective dosage of a long acting insulin, wherein said therapeutically effective dosage of said long acting insulin reduces said risks.

The present invention relates to the use of a long-acting insulin, in particular insulin glargine, in a method of reducing the risk of progression to type 2 diabetes in a patient, a method of reducing the risk of a new angina in a patient and a method of reducing the risk of a microvascular event in a patient comprising administering to said patient in need thereof a therapeutically effective dosage of a long acting insulin, wherein said therapeutically effective dosage of said long acting insulin reduces said risks.

Basal pancreatic insulin secretion is responsible for maintaining fasting plasma glucose (FPG) levels below 5.6 mmo/l (100 mg/dl) in normal individuals, and an elevated FPG level signifies that there is insufficient endogenous fasting insulin secretion to overcome underlying insulin resistance. This metabolic abnormality progresses with time and is reflected in progressively higher glucose and HbA1c levels. It and its progression are also risk factors for cardiovascular outcomes regardless of the presence or absence of diabetes [1, 2, 3, 4, 5, 6, 7]. They are also risk factors for incident diabetes in people with impaired fasting glucose or impaired glucose tolerance.

Despite the link between elevated glucose levels and cardiovascular outcomes, large outcomes trials of more versus less intense glucose lowering with insulin plus other glucose lowering drugs have not observed a clear cardiovascular benefit [8] and one of these trials noted increased mortality [9]. Moreover, basal insulin was used in both treatment arms in these trials so no conclusions regarding its isolated cardiovascular effects could be drawn. Of note, the trial with the biggest contrast in insulin use was conducted in people with newly diagnosed diabetes and reported a 15 and 13% reduction in myocardial infarction and death [10] respectively during an extended follow-up. However this trial was not restricted to people at high risk for cardiovascular outcomes and normal fasting glucose levels were not achieved and maintained during the trial in the treatment group.

These results evidence that insulin itself may have cardioprotective effects [11, 12, 13], the availability of a long-acting insulin preparation with a predictable duration of action and low risk of hypoglycemia, and evidence that exogenous insulin therapy may slow the decline in pancreatic dysfunction with time [14, 15, 16]. The Outcome Reduction with an Initial Glargine Intervention (ORIGIN) trial was a large international multicentre randomized controlled trial designed to explicitly test this possibility in people with IFG, IGT or early diabetes and additional cardiovascular risk factors [17].

### Diabetes Mellitus and Cardiovascular Disease

People with type 2 diabetes mellitus (DM) have an increased risk of atherosclerotic disease, including coronary heart disease, strokes, and peripheral vascular disease. Diabetes itself, and not just the associated risk factors of dyslipidemia, hypertension, and obesity contributes a major portion of this risk [18]. In particular, the level of hyperglycemia may play a key role. While the relationship of increased blood glucose to microvascular complications is well recognized, its relation to atherogenesis was, until recently, less well documented [19, 20, 21, 22]. A prospective, population-based study in middle-aged and elderly people in Finland with type 2 DM has shown a progressive relation between baseline fasting blood glucose (FBG) or HbA1c, and coronary heart disease mortality [23]. In the WESDR database, people diagnosed with diabetes at age 30 years or older had a statistically significant increase in mortality from vascular causes for every 1% increase in glycosylated hemoglobin [24]. The Islington Diabetes Survey found a progressive relationship between 2-hour postprandial glucose or HbA_{1c} and coronary heart disease, with the stronger association with the 2-hour glucose test [25]. In the San Antonio Heart Study, the level of hyperglycemia was a strong, independent predictor of all-cause and cardiovascular mortality [26].

### Impaired Glucose Tolerance, Impaired Fasting Glucose and Cardiovascular Risk

A growing body of evidence indicates that the increased risk for macrovascular complications associated with type 2 DM also extends to individuals with glucose abnormalities that do not meet the criteria for frank diabetes. The American Diabetes Association (ADA) defines IGT as a 2-hour glucose level (PPG) of 7.8-11.1 mM (140-199 mg/dL) after a 75 gram oral glucose load, with FPG levels below 7.0 mM (126 mg/dL). The ADA has recently recognized a new category of IFG, defined as a fasting plasma glucose of 6.1-6.9 mM (110-125 mg/dL) [27]. Cardiovascular disease is the leading cause of death in the U.S. population and is especially prevalent and predictive of mortality within the diabetic, IGT, and IFG populations [18]. An excess risk of cardiovascular events characterizes IGT and IFG as well as type 2 diabetes, and there is a continuum of risk beginning with the mildest degrees of abnormality of blood glucose and extending into the diabetic range [28, 29, 30]. This “dysglycemia” and its relation to cardiovascular disease is now the focus of much research interest [31].

The American Diabetes Association about 15 years ago lowered the fasting plasma glucose level at which diabetes is diagnosed from 140 to 126 mg/dL (7.8 to 7.0 mM). This was done because of the recognition that a fasting level of 126 mg/dL (7.0 mM) was more closely correlated to a 2 hour post-load level of 200 mg/dL (11.1 mmol)—the level above which the risk for microvascular disease begins to rise—than 140 mg/dL (7.8 mM) [27]. This new threshold was not, however, chosen because of any special significance with respect to macrovascular disease, which remains a leading cause of morbidity and mortality in people with IGT, IFG and diabetes.

The Hoorn study found an increased risk of all-cause and cardiovascular mortality with higher 2-hour post-load glucose values and increasing HbA_{1c} in a general population of men and women that included people with blood glucose levels extending from normal to the diabetic range [32]. In the EPIC Norfolk study, an increase of 1% in HbA_{1c} was associated with a 28% increase risk of death, and an increase of approximately 40% in cardiovascular or coronary heart disease mortality, in a cohort of 4662 men [33]. Although diabetic individuals were included in this trial, and diabetes was found to be an independent predictor of cardiovascular risk when evaluated separately from HbA_{1c} (another independent predictor), only HbA1c and not diabetes predicted CV death when both were included in the same analysis. This further illustrates the link between glucose elevations and CV risk, versus the presence or absence of diabetes. Similarly, a study in non-diabetic elderly women found that all-cause mortality and coronary heart disease were significantly related to fasting plasma glucose [34].

In a study from Oslo, non-diabetic men aged 40-59 years had a significantly higher cardiovascular mortality rate if their FPG was >85 mg/dL (4.7 mM) [35]. Long-term follow-up of several prospective European cohort studies has confirmed a higher risk of cardiovascular-related mortality in non-diabetic men with the highest 2.5% of values of FPG and 2-hour postprandial glucose [35]. A meta-regression analysis of data from 20 cohort studies found a progressive relationship between glucose levels and cardiovascular risk even below the cutoff points for diagnosis of DM [29]. Likewise, in the 23-year Paris Prospective Study of 7018 middle-aged nondiabetic men, increased fasting or 2-hour postprandial blood glucose was associated with increased total and coronary mortality in a graded, non-threshold relationship [36].

### Rationale for Study of Omega-3 Fatty Acids

Over the past 30 years, there has been a rapid expansion of knowledge on the effects of omega-3 polyunsaturated fatty acids (omega-3 PUFA, or n-3 PUFA) in coronary heart disease (CHD) [37]. Omega-3 PUFA include linolenic acid as well as eicosapentaenoic acid (EPA) and docosahexaenoic acid (DHA). Linolenic acid is an essential fatty acid provided by dietary sources including soybean and canola oils. EPA and DHA are also provided by dietary sources (eg, fish oils), but can also be derived by chain elongation and desaturation of linolenic acid.

Omega-3 PUFAs inhibit platelet aggregation and are anti-inflammatory [37]. Potential cardioprotective effects of n-3 PUFA which have been studied include decreasing circulating proatherogenic and prothrombotic factors such as arachidonic acid, thromboxane A₂, fibrinogen, platelet-derived growth factor, and platelet activating factor, as well as circulating triglycerides, chylomicrons, and Lp(a). Conversely, n-3 PUFA administration has been shown to increase the circulation of cardioprotective factors such as prostacyclin, tissue plasminogen activator, endothelium-derived relaxation factor, and HDL cholesterol [37].

Data from epidemiological studies [37] are mixed, but on the whole, suggest an association between n-3 PUFA intake and decreased risk of adverse CV events, particularly sudden death or death due to CHD. Rates of other CV events, such as MI, have been less closely linked to low blood levels or intake of n-3 PUFA.

A few important secondary intervention studies have been done, all in MI survivors, examining the impact of n-3 PUFA intake on reduction of CV risk. In the Diet and Reinfarction Trial (DART), a 29% decrease in all-cause mortality over 2 years was seen in men given a diet increased in fish, vs no diet advice [38]. In the Lyon Diet Heart Study, an n-3 PUFA-enriched diet conferred a 73% reduction in risk for CV death or nonfatal MI over a mean follow-up of 27 months [39]. Finally, the open-label GISSI-Prevenzione Trial [40] demonstrated a 15% relative risk reduction in a combined outcome of CV death, nonfatal MI, and nonfatal stroke in a population of 11,324 MI survivors who consumed 850-882 mg of n-3 PUFA per day, on average.

The overall benefits of n-3 PUFA treatment in GISSI were attributable to decreases in the risk of all-cause cardiovascular death, and of sudden death, with little impact on the incidence of MI or stroke.

Few adverse effects of n-3 PUFA have been demonstrated. Increases in blood glucose in diabetic participants, mild tendencies to bleeding, increased LDL concentrations, and increased PA1-1 levels have been noted in some trials. These effects have not been borne out in larger trials, and the LDL effects seem to be transient in longer studies (possibly related to the triglyceride-lowering effects of n-3 PUFA). A recent article [41] described n-3 PUFA as safe and effective in hypertriglyceridemic states, both primary and secondary (such as dysglycemia). A recent NIH workshop on the efficacy and safety of n-3 PUFA in people with diabetes concluded that further intervention studies of n-3 PUFA in the diabetic population are needed to clarify these issues.

Because of the aggregate evidence suggesting that increased n-3 PUFA intake can protect patients at risk for CV morbidity and mortality from future events, particularly CV death, this agent has been chosen as a separate treatment for the dysglycemic participants of the ORIGIN Study. Omega-3 PUFA may have a more profound effect in the setting of dysglycemia, in view of the lipid abnormalities and prothrombotic tendencies of the population, both of which may be favorably affected by n-3 PUFA augmentation.

### Rationale for the Study of Insulin Glargine

The ORIGIN study is a large-scale intervention trial of the use of insulin to decrease the risk of cardiovascular mortality and morbidity in a population of participants with impaired glucose tolerance (IGT), impaired fasting glucose (IFG), or early type 2 diabetes. This study has the acronym ORIGIN (Outcome Reduction with an Initial Glargine Intervention).

Although there is enhanced awareness of cardiovascular risk factors in the American population (e.g. regular surveillance and intervention for blood pressure and lipid abnormalities), until recently, the excess risk of cardiovascular disease associated with dysglycemia has received little recognition. Consequently, people with IFG or IGT are rarely treated with interventions aimed at reducing blood glucose levels. This is in part because mild hyperglycemia is often asymptomatic (as is the case for hypertension and hyperlipidemia), and because of the perceived risk of existing antihyperglycemic therapies for associated morbidity (e.g. the tendency of some agents to promote hypoglycemia). Additionally, there are no data to evaluate whether lowering blood glucose in those with IFG or IGT will decrease microvascular disease.

Evidence has provided support for a beneficial effect of insulin treatment started at the time of a myocardial infarction. In the DIGAMI study [42], diabetic patients hospitalized with acute MI were allocated to receive an IV insulin-glucose infusion in-hospital followed by intensive chronic outpatient treatment with insulin. Compared to standard treatment, the insulin-treated participants had a significant 28% reduction of all-cause mortality. Most of these deaths were cardiovascular in etiology. The most striking reductions in mortality were seen in the subset of patients without prior insulin treatment, with low cardiovascular risk pre-MI. In those individuals significant survival differences were even seen pre-discharge (while still in hospital post-MI), and enhanced survival in the same cohort was also observed during long-term follow-up.

Part of the benefit of insulin treatment was likely due to improved long-term glycemia post-MI, but the rapid benefits in-hospital suggest that other, more acute, effects of insulin besides long-term glycemic control may have played a role. These may include improved platelet function, decreased PAI-1 levels, and insulin-mediated reductions in circulating free fatty acid levels with consequent improved dyslipidemia and decreased myocardial oxygen requirement. Chronic insulin therapy may thus provide a level of protection against the cumulative deleterious effect of even subacute episodes of ischemia, and on the progression of atherosclerosis.

A recent study from Belgium [43] reinforces the beneficial role of insulin treatment in critically ill subjects. In this trial, critical-care post-surgical patients with random blood glucose values greater than 110 mg/dL (6.1 mM) were treated while in the ICU either with an insulin infusion to lower blood glucose to the 80-110 mg/dL (4.4-6.1 mM) range; or to receive insulin infusions only if blood glucose exceeded 215 mg/dL (11.9 mM), to reduce the blood glucose to between 180 and 200 mg/dL (10-11.1 mM). Twelve-months follow-up showed a significant reduction in overall mortality in the intervention group (8.0%, versus 4.6% in the control group); most of the benefit was attributable to the cohort of subjects who were in the ICU for 5 days or more. In-hospital mortality, septicemia, acute renal failure and hemodialysis incidence, and transfusion requirements were also significantly reduced in the intervention group versus the control group.

The use of exogenous insulin in an IGT, IFG, or diabetic population might confer several potential metabolic and cardiovascular benefits [44, 45, 46, 47, 48]:
1. The fact that it is finely titratable and durable (compared to oral antidiabetic agents) may translate into a powerful effect to delay the exposure of target tissues to toxic levels of glycemia.
2. Insulin-mediated suppression of circulating free fatty acids (FFA) will:
   reduce VLDL synthesis and improve lipoprotein patterns (i.e. lower triglycerides, and increase HDL-C);
   reduce lipotoxicity at the level of the beta cell and at insulin's target tissues;
   reduce obligatory oxidative metabolism in ischemic myocardium.
3. Exogenous insulin will prevent metabolic decompensation due to stress, that is either mild and frequent (i.e. daily stresses and minor illness or injury), or severe and less common (i.e. major injury, illness, surgery, vascular events). These stress events would normally suppress endogenous insulin responses even when a pharmacologic secretagogue or sensitizer is present; exogenous, injected insulin cannot be suppressed in such a way.
4. Nitric oxide-mediated vasodilation and endothelial function are abnormal in those with IFG, IGT, or diabetes. Additionally, markers of endothelial inflammation are increased. These abnormalities are all improved by insulin treatment [49, 50, 51, 52].
   Insulin glargine ((Gly A21) Arg (B31) Arg (B32) human insulin) is an approved insulin analog characterized by a smooth, 24-hr glucose lowering effect without a definite peak. As a basal insulin supplement, insulin glargine is capable of being finely titrated, and has no dose ceiling other than that dictated by its glucose-lowering action. A double-blind study (HOE901/1021) was conducted to explore the safety and feasibility of insulin glargine administration to people with IGT, IFG, or early diabetes. Participants were confined to a treatment center for two weeks, during which time they received a calorie-restricted diet appropriate for their degree of obesity, and insulin glargine or placebo was titrated to effect (FPG of 80-95 mg/dL, 4.4-5.3 mM). Moderate exercise challenges were performed at the beginning and end of the study. Thirteen participants with either IGT, IFG, or early diabetes received insulin glargine and 4 were given placebo insulin. Two of these 13 insulin glargine subjects experienced hypoglycemia, versus none of the placebo-treated subjects. All the episodes were mild, generally occurred prior to lunch or supper (but not in response to exercise), and resolved rapidly on snacking or eating. Based on this pilot study, insulin glargine presents a low risk for hypoglycemia in this population even when diet prescriptions for calorie restriction are implemented. The pilot study opened the way for the full-scale investigation in ORIGIN of the safety and efficacy of insulin glargine in the chronic intensive treatment of hyperglycemia across the whole dysglycemic population.

### Rationale for Extending the Origin Trial

By the spring of 2008, several studies had reported new data pertaining to the effect of glucose-lowering interventions in people with type 2 diabetes. These studies include: a) the passive follow-up of the United Kingdom Prospective Diabetes Study (UKPDS) of people with newly diagnosed diabetes [53], b) the ACCORD study of 10251 people with established diabetes (mean duration 10 years) and high CV risk [54], c) the ADVANCE study of 11140 people with established diabetes (mean duration 8 years) [55] and high CV risk; d) the VA diabetes trial (VADT) of 1791 people (mainly men) with established diabetes and high CV risk (not yet published); and d) the PROACTIVE study [56] which tested the effect of pioglitazone versus placebo in 5238 people with established diabetes (mean duration 8 years) and high CV risk. All of these findings were reported after ORIGIN recruitment had been completed, and with the exception of the PROACTIVE study reported the effect of more versus less intensive glucose lowering on CV outcomes.

These studies' data are generally consistent with the hypothesis that a gluco-metabolic intervention may reduce CV outcomes in people with type 2 diabetes. Specifically, the significant 15% lower rate of myocardial infarction and 13% lower risk of death after 17 years of follow-up of the UKPDS participants (and 8.5 years after the active treatment phase ended) [53], a significant 24% reduced risk of myocardial infarction and a trend suggesting a reduced composite CV outcome during 3.5 years of follow-up in the ACCORD trial [54], a significant 17% reduced risk of myocardial infarction, a trend suggesting a reduced composite CV outcome in the VADT [57], and a 16% reduction in myocardial infarction, stroke or CV death together with a trend suggesting a reduced primary composite CV outcome in PROACTIVE during 2.9 years of follow-up [55], all support this possibility. Unfortunately, the truncated follow-up of the ACCORD study (due to the increased mortality in the treatment group) precluded the ability to determine if there is a long-term benefit. Moreover, the fact that it took approximately 3 of the 5 years of follow-up to achieve a stable (but modest) HbA1c between-group contrast in ADVANCE, the small sample size and low power of the VADT, and the short follow-up of the PROACTIVE trial reduced the power of these studies to clearly detect a benefit,

Indeed, inspection of the event curves for these trials as well as the long-term follow-up of the DCCT in people with type 1 diabetes study [58] suggest that any CV benefit of a glucose-lowering intervention requires at least 3 years after a stable glycemic or therapeutic contrast has been achieved to begin to become apparent, and more than 5 years to be clearly detectable. For example, in the UKPDS obesity study, the effect of metformin on the risk of myocardial infarction and death became apparent only after 4-5 years [59].

These trials also indicated that a gluco-metabolic intervention may be more effective in people with earlier or less advanced diabetes. Thus the UKPDS identified a long-term CV benefit in people with newly diagnosed diabetes [53], and the ACCORD trial reported a clear reduction in the CV composite outcome exceeding 20% in the prospectively identified subgroup of participants whose baseline HbA1c level was less than 8% [54]. Finally, data presented by the VADT investigators [57] suggested that participants with a shorter duration of diabetes may realize a greater CV benefit from a gluco-metabolic intervention.

The ORIGIN trial had a mean follow-up of 3.5 years as of July 2008 and was originally scheduled to end after a median follow-up of approximately 4.5 years. It has several unique features that address many of the questions raised by the aforementioned trials [60]:
a) participants at high CV risk but at a much earlier stage of dysglycemia are being studied;
b) participants had lower baseline HbA1c levels with either “prediabetes”, newly diagnosed diabetes, or a relatively short 5 year mean duration of diabetes;
c) it is designed to test the effect of insulin-replacement therapy (i.e. insulin-mediated normoglycemia as measured by the fasting plasma glucose) versus usual care, and not to test the effect of a lower versus higher HbA1c level;
d) insulin replacement therapy also lowers free fatty acid levels, which are themselves significant risk factors for CV outcomes.
e) it continues to be monitored by an experienced IDMC which has not raised any safety concerns to date.

In summary, the following considerations all supported a 24 month extension of ORIGIN:
a) Recent studies suggest that if there is a CV benefit to a gluco-metabolic intervention it will take up to 5 years after a stable contrast has emerged to be detectable.
b) The extension will test more than 5 years of a stable contrast.
c) The extension will allow further accrual of events and will increase power.
d) ORIGIN is the only trial of an insulin-mediated intervention in people with early dysglycemia, who represent a large number of people at high risk for CV outcomes.
e) The study hypotheses remain unchanged.
f) The plan to extend the study is only based on the above considerations and not on any analyses of interim outcome data, which have only been seen by the IDMC.

### Study Objectives

### Primary Objectives

To determine whether insulin glargine-mediated normoglycemia can reduce CV morbidity and/or mortality in people at high risk for vascular disease with either IFG, IGT, or early type 2 diabetes;

To determine whether omega-3 polyunsaturated fatty acids (n-3 PUFA) can reduce cardiovascular mortality in people with IFG, IGT, or early type 2 diabetes.

### Secondary Objectives

The secondary objectives of the insulin glargine study are to determine if insulin glargine-mediated normoglycemia can reduce:
total mortality (all causes);
the risk of diabetic microvascular outcomes (composite outcome: kidney or eye events);
the rate of progression of IGT or IFG to type 2 diabetes.

The secondary objectives of the omega-3 PUFA study are to determine if n-3 PUFA reduce:
major vascular events (a composite of: cardiovascular death; myocardial infarction; or stroke)
all-cause mortality
A composite of sudden unexpected death, non-sudden arrhythmic death, unwitnessed death, or resuscitated cardiac arrest

In the following there are definitions provided regarding cardiovascular efficacy outcomes.

Cardiovascular Death is defined as any of the following:

Sudden Unexpected Death: defined as death that occurred suddenly and unexpectedly in which the death is witnessed and the time of death is known: witnessed death due to:
An identified arrhythmia (ECG or at least monitor recording, or monitor-witnessed arrhythmia either by a medic or a paramedic)
Cardiac arrest or cardiovascular collapse in absence of premonitory heart failure or myocardial infarction or other modes of death.
Patients resuscitated from a sudden cardiac arrest who later die of the sequelae of the event, or patients who die during an attempted resuscitation.

Non-sudden Arrythmic Death: defined as death due to documented arrhythmia when death is not sudden and not unexpected and is not associated with evidence of myocardial ischemia (e.g., patient with recurrent tachyarrhythmia or bradyarrhythmia who died 6 hours after admission to the hospital).

Unwitnessed Death: Death that occurred in which the time of death is unknown. In this case, the interval between the time the patient was last seen and the time the death became known will be recorded. In some circumstances, can be considered to be unexpected.

Fatal Myocardial Infarction (MI): Fatal myocardial infarction may be adjudicated in any one of the following three scenarios:
Death occurring after a documented myocardial infarction in which there is not conclusive evidence of another cause of death. Patients who are being treated for myocardial infarction and who have a sudden death as the terminal event related to the MI will be classified as having a myocardial infarction-related death.
Autopsy evidence of a recent infarct with no other conclusive evidence of another cause of death.
A fatal myocardial infarction may be adjudicated for an abrupt death that has suggestive criteria for an infarct but does not meet the strict definition of a myocardial infarction. The suggestive criteria are presentation of chest pain and one of the following:
ECG changes indicative of a myocardial injury or
Abnormal cardiac markers without evolutional changes (i.e., patient died before a subsequent draw) or
Other evidence of new wall motion abnormality

Heart Failure Death: death due to heart failure, with clinical, radiological, or postmortem evidence of heart failure but without evidence of other cause such as ischemia, infection, dysrhythmia. Cardiogenic shock to be included.

Death after Invasive Cardiovascular Intervention: includes death occurring within 30 days of cardiovascular surgery, or within 7 days of cardiac catheterization, arrhythmia ablation, angioplasty, atherectomy, stent placement, or other invasive coronary or peripheral vascular intervention

Death Due to Stroke: Death due to stroke and occurring within 30 days of signs/symptoms of stroke

Other Cardiovascular Causes of Death: other vascular events, including pulmonary emboli and ruptured abdominal aortic aneurysm

Presumed Cardiovascular Death: death suspicious of cardiovascular death with supporting clinical evidence that may not fulfill other criteria (e.g., patient with chest pain typical for MI, but without ECG or enzyme documentation that fulfills MI criteria)

Death from Unknown Cause: qualifies as a cardiovascular event unless clear evidence of extraneous disease exists

Non-cardiovascular Death is defined as any death for which clear evidence of a non-cardiovascular cause exists. Categories of non-cardiovascular death include:

### Malignancy

Gastrointestinal malignancy
Lung Malignancy
Breast Malignancy
Prostate Malignancy
Brain Malignancy
Skin Malignancy
Multi-site malignancy
Genito-urinary malignancy
Other malignancy (specify)

### Other Non-Cardiovascular Death not Due to Malignancy

Non-fatal Myocardial Infarction is defined as any of the following: Non-Procedural MI:

### EITHER

Ischemic Symptoms: (pain, dyspnea, pressure) at rest or accelerated ischemic symptoms, either of which lasts ≧10 minutes that the investigator determines is secondary to ischemia

### OR

ECG changes consistent with infarction:
New significant Q waves (or R waves in V1-V2) in two contiguous leads in the absence of previous LVH or conduction abnormalities
Evolving ST-segment to T-wave changes in two or more contiguous leads
Development of new left bundle branch block
ST segment elevation requiring thrombolytics or PCI

### AND

### Cardiac Markers:

If troponin is drawn:
Any combination of markers where troponin result is in necrosis range.
If troponin is not in the necrosis range, at least one other marker must be 2×ULN.
If troponin given in ranges, the diagnostic lower limit for MI will be considered the lowest value in the range indicative of necrosis.
   If troponin is not drawn:
If both CK and CKMB are drawn, both values must be ≧ULN.
If both CK and CKMB are drawn and CK value is <ULN, CKMB must be ≧1.5×ULN.
If only CKMB is drawn, must be 1.5×ULN.
If only CK is drawn, must show serial changes of 2×ULN
   Other cardiac markers: These markers would include SGOT, LDH, or myoglobin and could be used if they are drawn to rule out myocardial injury. In this case, they must demonstrate serial changes (≧2×ULN) and should only be used when cardiac-specific markers are unavailable.

### Procedural MI:

Post-PCI MI

### EITHER

New pathologic Q waves (may also have other clearly documented wall motion abnormalities other than septal)

### OR

Cardiac Markers (within 24 hours of procedure): Marker ≧3×ULN and ≧50% above last measurement if last measure was ≧ULN
Post-CABG MI

### EITHER

New pathologic Q waves (may also have other clearly documented wall motion abnormalities other than septal)

### OR

CKMB (within 24 hrs of procedure): CKMB≧5×ULN and 50% above last measurement if last measure was ≧ULN

### Silent MI:

It is acknowledged that there are instances where myocardium necrosis attributed to myocardial infarction occurs which is clinically unrecognized. If the investigator (based on review of the clinical status and ECGs) feels that this occurred, he/she should submit information supporting the diagnosis of a clinically unrecognized myocardial infarct. Support would require at least paired ECGs showing new and significant Q-waves not attributed to intraventricular conduction defect, left ventricular hypertrophy, pre-excitation syndrome, or electronic pacer. In addition, confirmation may be achieved by echocardiographic or other evidence of new regional wall motion abnormalities. The Event Adjudication Committee (EAC) will evaluate clinically reported events in a blinded fashion and ascertain whether they have sufficient information to concur that a significant event, which was clinically unrecognized, has occurred. The timing of that event would be the earliest ECG showing new Q-waves.

### Non-Fatal Stroke

Stroke is defined as the presence of acute focal neurological deficit (except for subarachnoid hemorrhage which may not be focal) thought to be of vascular origin with signs or symptoms lasting greater than 24 hours. On the basis of clinical symptoms, autopsy and/or CT/MRI/other imaging modality, strokes will be classified as:

### Definite or Probable Ischemic Stroke

Stroke with CT/MRI/other imaging modality performed within 3 weeks that is either normal or shows infarct in the clinically expected area. Subgroups of ischemic stroke include:
Lacunar infarct—cerebral infarction with:
   Consciousness and higher mental functions maintained
   One of the typical lacunar syndromes such as pure motor stroke, pure sensory stroke, sensori-motor stroke, or ataxic hemiparesis.
   CT/MRI/other imaging modality performed within 3 weeks that is either normal or shows a small infarct in the basal ganglia, internal capsule, medulla, or pons.
Cardioembolic infarct—Cerebral infarction with:
   Absence of lacunar characteristics
   No definite evidence of large artery disease in the neck
   Major cardioembolic source present (e.g., atrial fibrillation, myocardial infarction in the last 6 weeks, cardiomyopathy, endocarditis, or prosthetic heart valve)
Large artery infarct
   Absence of lacunar characteristics
   No major cardioembolic source present
   Evidence of large artery disease in the neck (eg. a bruit, or duplex scan evidence of a stenosis of more than 50%)
Unclassified infarct: Cerebral infarction that is not lacunar, cardioembolic or large artery in origin (including a stroke with more than one potential cause)

### Definite Hemorrhagic Stroke:

Definite stroke with cerebral hemorrhage confirmed by CT/MRI/other imaging modality or autopsy. Does not include hemorrhage secondary to cerebral infarct, trauma, hemorrhage into a tumor, or vascular malformation.

### Definite Stroke, Type Uncertain:

Definite stroke that does not meet the above criteria for ischemic stroke or hemorrhage.

### Subarachnoid Hemorrhage:

Typical clinical syndrome of sudden onset headache, with or without focal signs, and CT/MRI/other imaging modality or cerebrospinal fluid evidence of bleeding primarily in the subarachnoid space.

Revascularization Procedures include any of the following:
PTCA (balloon)
PTCA with stent
Other PCI
CABG
Carotid Angioplasty with Stent
Carotid Endarterectomy
Peripheral Angioplasty with or without Stent
Peripheral Vascular Surgery (including abdominal aortic aneurysm repair)
Limb Amputation (including partial or digit amputation) due to vascular disease

### Resuscitated Cardiac Arrest

Resuscitated cardiac arrest is defined as sudden cardiac arrest, with or without premonitory heart failure or myocardial infarction, following which the patient is resuscitated by cardioversion, defibrillation or cardiopulmonary resuscitation. This definition excludes known transient losses of consciousness such as seizure or vasovagal episodes that do not reflect significant cardiac dysfunction. In order to meet the criteria for this event the patient should also gain a reasonable amount of consciousness after the resuscitation without the aid of artificial life support.

### Hospitalization for Cardiovascular Causes

All hospitalizations will be encoded by the Data Center using the MedDRA dictionary. Cardiovascular hospitalizations will be defined as any hospitalization that is encoded by the Data Center to a term in the MedDRA dictionary that maps to the cardiovascular body system.

### Hospitalization for Heart Failure

Hospitalization for heart failure is defined as a hospitalization for congestive heart failure or attendance in an acute care setting (Emergency Room) for administration of intravenous diuretic, escalation of diuretic doses and/or inotropes, and confirmed by chest x-ray.

### New Angina

New onset of typical angina with documented ischemia by stress testing (ECG, ECHO, or nuclear)

### Worsening Angina

Known angina increasing in frequency, duration, and/or severity, and requiring hospitalization and/or increased anti-anginal medication

### Unstable Angina

Unstable angina is defined as ischemic symptoms: (pain, dyspnea, pressure) at rest or accelerated ischemic symptoms, either of which lasts 10 minutes, that the investigator determines is secondary to ischemia

### AND

Ischemic ECG changes as compared to most recent ECG or during the previous stable phase:
≧0.5 mm transient ST segment depression in two contiguous limb or precordial leads
≧1 mm transient ST elevation of two contiguous leads (or ST depression in V1 or V2)
≧2 mm transient T wave change in two or more contiguous leads

### OR

### Cardiac Markers:

Cardiac marker suggestive of myocardial injury, ≧ULN but not sufficient for MI criteria. If troponin is used, must be in the “suggestive” (middle) range for necrosis.

### Vascular Amputation

Amputation of a limb or part of a limb secondary to vascular insufficiency

### Cognitive Function

Defined by serial cognitive testing (e.g., mini-mental status examination [MMSE]).

In the following there are definitions provided regarding microvascular outcome variables.

The composite microvascular outcome will be met by the development of any of the following:
Doubling of serum creatinine from study baseline (screening value)
Albuminuria progression, defined as a change from normoalbuminuria to either microalbuminuria or clinical proteinuria, or from microalbuminuria to clinical proteinuria using the definitions in the attached table.

Definitions of Normoalbuminuria, Microalbuminuria,
and Clinical Proteinuria

| | Normo- | | Micro- | | Clinical | |
|---|---|---|---|---|---|---|
| Method | albuminuria | | albuminuria | | Proteinuria | |
| Spot Urine for | <30 | mg/g | ≧30 | mg/g | ≧300 | mg/g |
| Albumin: | | | <300 | mg/g | | |
| Creatinine | | | | | | |
| Ratio | | | | | | |
| Timed Urine | <20 | μg/min | ≧20 | μg/min | ≧200 | μg/min |

| | | | | |
|---|---|---|---|---|
| Collection for | or | <200 | μg/min | or |

| | | | |
|---|---|---|---|
| Albumin | <30 | mg/24 hrs | or |

| | | | | |
|---|---|---|---|---|
| Excretion | ≧30 | mg/24 hrs | ≧300 | mg/24 hrs |
| | <300 | mg/24 hrs | | |

| | | | | |
|---|---|---|---|---|
| Timed Urine | N/A | N/A | ≧500 | mg/24 hrs |
| Collection for | | | | |
| Total Protein | | | | |
| Excretion | | | | |

Requirement for renal replacement therapy (eg, dialysis, renal transplant), or death due to renal failure
Use of retinal photocoagulation or vitrectomy for diabetic retinopathy, including macular edema

Diabetes mellitus is associated with an increased incidence of bone fractures, and vertebral fractures result in a decrease in height.

The waist-hip ratio (WHR) has been used as an indicator or measure of the health of a person, and the risk of developing serious health conditions.

By the Origin study it has been surprisingly found that although there was no statistically significant difference in mortality or microvascular outcomes, there is a trend that treatment with insulin glargine is beneficial with regard to microvascular outcomes. Moreover, participants without diabetes at randomization who were allocated to insulin glargine were significantly less likely to develop protocol-defined diabetes than standard care participants. Also, under an early intervention with insulin glargine a highly significant effect on the development of new angina was detected.

The results of the ORIGIN study were obtained with the long-lasting insulin glargine. Respective studies with other long-acting insulins like insulin detemir (Levemir®) and insulin degludec (Tresiba®) lead to comparable results.

Therefore, an embodiment of the invention is a method of reducing the risk of progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin reduces the risk of progression to type 2 diabetes in said patient.

A further embodiment of the invention is a method of reducing the risk of a new angina in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receive an oral antidiabetic agent, comprising administering to said patient a therapeutically effective dosage of a long acting insulin, wherein said therapeutically effective dosage of said long acting insulin reduces the risk of a new angina.

A further embodiment of the invention is a method of reducing the risk of a microvascular event in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receive an oral antidiabetic agent, comprising administering to said patient a therapeutically effective dosage of a long acting insulin, wherein said therapeutically effective dosage of said long acting insulin reduces the risk of a microvascular event.

A further embodiment of the invention is a method for preventing the progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin reduces the risk of progression to type 2 diabetes in said patient.

A further embodiment of the invention is a method for preventing a new angina in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receive an oral antidiabetic agent, comprising administering to said patient a therapeutically effective dosage of a long acting insulin, wherein said therapeutically effective dosage of said long acting insulin reduces the risk of a new angina.

A further embodiment of the invention is a method for preventing a microvascular event in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receive an oral antidiabetic agent, comprising administering to said patient a therapeutically effective dosage of a long acting insulin, wherein said therapeutically effective dosage of said long acting insulin reduces the risk of a microvascular event.

A further embodiment of the invention is a method delaying the progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin delays the progression to type 2 diabetes in said patient.

A further embodiment of the invention is as described above, wherein the microvascular event is a clinical microvascular event, in particular wherein the microvascular event is selected from a group comprising neuropathy, retinopathy and nephropathy, preferably wherein the nephropathy is characterized by renal failure, end-stage renal disease, or renal death.

A further embodiment of the invention is a method for reducing the risk for requiring treatment by laser surgery or vitrectomy in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting reduces the risk for requiring treatment by laser surgery or vitrectomy in said patient.

A further embodiment of the invention is a method for reducing doubling of baseline serum creatinine in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces doubling of baseline serum creatinine in said patient.

A further embodiment of the invention is a method for reducing the risk of cognitive impairment in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces the risk of cognitive impairment in said patient, in particular wherein the patient scores 24 or less in the Mini-Mental Status Exam (MMSE).

A further embodiment of the invention is a method for lowering the triglyceride concentration in the blood in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin lowers the triglyceride concentration in the blood in said patient.

A further embodiment of the invention is a method for lowering the cholesterol concentration in the blood in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin lowers the cholesterol concentration in the blood in said patient.

A further embodiment of the invention is a method of reducing the risk of a microvascular event or a method for preventing a microvascular event as both described above, wherein the patient has a HbA1c≧6.4 prior to administering the long-acting insulin.

A further embodiment of the invention is a method of reducing the risk of a microvascular event or a method for preventing a microvascular event as both described above, wherein the patient had a history of atrial fibrillation prior to administering the long-acting insulin, in particular wherein the microvascular outcome is a clinical microvascular outcome or a laboratory-based microvascular outcome, preferably wherein the microvascular outcome is a composite of: laser surgery or vitrectomy or blindness for diabetic retinopathy; development of renal death or the need for renal replacement treatment (dialysis or transplantation); doubling of serum creatinine; or progression from lesser to greater severity of microalbuminuria.

A further embodiment of the invention is a method as described above, wherein the long-acting insulin is selected from a group comprising insulin glargine, insulin detemir and insulin degludec; preferably selected from a group comprising insulin glargine.

A further embodiment of the invention is an article of manufacture comprising
a packaging material;
a long-acting insulin; and
a label or package insert contained within the packaging material indicating that patients receiving the treatment with the long-acting insulin can be treated by a method as described above.

A further embodiment of the invention is an article of manufacture comprising
a packaging material;
insulin glargine; and
a label or package insert contained within the packaging material indicating that patients receiving the treatment with the long-acting insulin can be treated by a method as specified above, wherein in such treatment the risk for cardiovascular outcomes, all-cause mortality or cancer is not altered when compared to standard glucose lowering therapy, in particular wherein the risk for cancer is not altered when compared to standard glucose lowering therapy with regard to any organ-specific type of cancer, in particular wherein the long-acting insulin is selected from a group comprising insulin glargine, insulin detemir and insulin degludec; preferably selected from a group comprising insulin glargine.

### BRIEF DESCRIPTION OF THE DRAWINGS

FIG. 1: Flow of study participants from screening to analysis

FIG. 2: Forest plot of hazard ratios of the primary, secondary, and other ORIGIN outcomes

FIG. 3: Proportion of participants who experienced the co-primary composite outcome of myocardial infarction, stroke, or cardiovascular death (Panel A), these outcomes plus revasculatization or heart failure hospitalization (Panel B), or mortality (Panel C)

FIG. 4: Forest plot of odds of newly diagnosed diabetes. Row 1 illustrates the odds of new diabetes as defined in the protocol; row 2 illustrates the odds of diabetes after a s second glucose tolerance test (done only on those without diabetes after the 1^{st} test) and row 3 illustrates both confirmed diabetes and diagnoses of diabetes that were suspected but not confirmed.

FIG. 5: Fasting plasma glucose and A1C responses by treatment assignment. Open circles and broken lines denote standard therapy; solid circles and lines denote treatment with insulin glargine. The subgroups by glycemic status at entry are shown separately: dysglycemia without diabetes in A and C, diabetes in B and D. Medians are shown. Numbers of measurements at each time-point for standard therapy and glargine appear at the bottom of each panel. Std=Standard; Gla=Glargine; End=end of treatment.

FIG. 6: Percentages of participants with A1C<7.0% or 6.5% by treatment assignment over time. Open circles and broken lines denote standard therapy; solid circles and lines denote treatment with insulin glargine. The subgroups by glycemic status at entry are shown separately: dysglycemia without diabetes in A and C, diabetes in B and D.

FIG. 7: Forest plot of Odds Ratios (OR) for maintaining mean A1C<6.5% over 5 years with glargine versus standard therapy, by subgroups independently associated (p<0.05) with this outcome in the logistic regression model shown in Table 9. P values for interaction between the effect of treatment assignment and each subgroup are shown.

The invention is described in the following by examples.

### Example 1

### Investigational Plan

The ORIGIN study was an international, multicenter, randomized, open-label (for insulin glargine versus standard care), double-blind (for omega-3 PUFA versus placebo), 2×2 factorial design study to evaluate whether patients with IGT, IFG, or early T2DM, who were at high risk for macrovascular events, could be safely treated with insulin glargine and omega-3 PUFA, and if either insulin glargine-mediated normoglycemia and/or omega-3 PUFA reduce or prevent CV morbidity and/or mortality. Patients were randomized to either receive insulin glargine treatment as a titrated regimen which targeted fasting plasma glucose (FPG) of 95 mg/dL or standard care according to current guidelines for dysglycemia accompanied by appropriate lifestyle modifications. Patients were also independently randomized to receive either ethyl esters of omega-3 PUFA or matching placebo.

The study consisted of a 2-year recruitment period, and was originally planned to also include an average of 4 years of treatment and follow-up. After the study was extended by 24 months, it was estimated that the mean duration of treatment and follow-up would increase to approximately 6.5 years and the total duration of the study to approximately 7.5 years (2 years recruitment period and at least 5.5 years follow-up after the last patient randomization).

However, the study was event-driven, and its actual duration was to be based on the number of observed events. The study ended when a prespecified total number of primary outcomes (2 200 patients having experienced at least one component of the primary outcome) needed for a sufficient statistical power to test the insulin glargine group against the standard care group was achieved.

If this event total had not been achieved after 7.5 years, the IDMC could have recommended to the Steering Committee that the follow-up of patients be extended until the prespecified number has been reached.

Approximately twelve thousand five hundred (12 500) dysglycemic patients with evidence of CV disease who were at high risk for future CV events were enrolled. The study population comprised the following three groups:
Patients with IFG and/or IGT (ie, prediabetic patients);
Patients with new or previously diagnosed T2DM who had been taking no pharmacotherapy for hyperglycemia for at least the preceding 10 weeks;
Patients with established T2DM who had been taking one oral antidiabetic drug (OAD) at stable dose for at least the preceding 10 weeks. Patients taking combination products containing two or more OADs were not eligible.

Patients were to be randomly assigned to receive either insulin glargine treatment or standard care for their dysglycemia. Patients randomized to the insulin glargine group received Lantus® (insulin glargine 100 U/mL solution) once daily (QD) by subcutaneous (SC) injection in a titrated regimen targeting an FPG of ≧95 mg/dL (5.3 mmol/L). Nondiabetic patients randomized to standard care were followed for the development of diabetes, and were encouraged to continue to modify diet and physical activity levels. Blood glucose management of diabetic patients (or nondiabetic patients who developed diabetes during the study) randomized to standard care was to be performed according to current (at that time) guidelines. All patients were to be encouraged to appropriately modify their lifestyle.

Patients were also to be independently randomly assigned to receive either Omacor® (ethyl esters of omega-3 PUFA), or matching placebo. Randomization to insulin glargine versus standard care and omega-3 PUFA versus matching placebo could occur at separate visits for some patients, as omega-3 PUFA and matching placebo were not available at the same time as insulin glargine at some sites. Thus some patients were randomized to insulin glargine versus standard care, and begin receiving their assigned treatment from among these two, before being randomized to receive omega-3 PUFA versus matching placebo. In the opinion of the Steering Committee, the delay in this omega-3 randomization was not to affect patient safety or well-being, and was to only marginally affect the power of the study to answer the omega-3-related study questions.

In this event-driven study, patients were enrolled for approximately 7 years, including:
Screening: up to more than 10 weeks (a qualifying oral glucose tolerance test [OGTT] could have been obtained up to 4 weeks prior to signing informed consent at the screening visit; taking OAD for at least 10 weeks at the time of screening or for 10 weeks prior to hospitalization if identified while hospitalized for a CV event);
Run-in: 4 to 10 days (for a successful completion of home glucose monitoring [HGM] and self-injection of the insulin glargine placebo [insulin pen cartridges containing physiologic saline]);
Treatment and follow-up: a mean of 6.5 years (ranging from 5.5 to 7.5 years) from randomization till the end-of-usual follow-up [EUF];
Post-EUF OGTT: 3 to 14 weeks (for OGTT in selected patients who were not classified as having had diabetes by the EUF).

Routine visits were to occur at 2, 4, 8, and 16 weeks following randomization, then every four months for the rest of the study, for all patients.

### Example 2

### Selection of Study Population—Inclusion Criteria

1. Individuals with either IFG and/or IGT, or early diabetes, as defined below.
   A Impaired glucose tolerance (IGT), defined as a PPG value ≧140 and <200 mg/dL (ie, ≧7.8 and <11.1 mmol/L), with a FPG <126 mg/dL (7.0 mmol/L).
   OR
   B Impaired fasting glucose (IFG), defined as an FPG ≧110 and <126 mg/dL (≧6.1 and <7 mmol/L), without diabetes mellitus (PPG must be <200 mg/dL [11.1 mmol/L]).
   OR
   C Early type 2 diabetes, defined as a FPG 26 mg/dL (7.0 mmol/L) or a PPG of ≧200 mg/dL (11.1 mmol/L), or a previous diagnosis of diabetes, and either:
      on no pharmacological treatment (while ambulatory for at least 10 weeks prior to screening, with screening glycated hemoglobin <150% of the upper limit of normal (ULN) for the laboratory (eg, <9% if the ULN is 6%)
      OR
      taking one OAD from among sulfonylureas (SU), biguanides, thiazolidinediones (TZDs), alpha-glucosidase inhibitors (AGIs), and meglitinides (MGTs) at a stable dose while ambulatory for at least 10 weeks at the time of screening (or for the 10 weeks prior to hospitalization if identified while hospitalized for a CV event), with screening glycated hemoglobin <133% of the ULN for the laboratory (eg, <8% if the ULN is 6%) if taking this medication at half-maximum dose or greater, and glycated hemoglobin <142% of the ULN for the laboratory (eg, <8.5% if the ULN is 6%) if taking this medication at less than half-maximum dose. Individuals taking combination products containing two or more OADs were not eligible.
2. Men or women aged 50 years and older.
3. Participants must be at risk for cardiovascular disease, based on satisfying one or more of the following criteria [MT: Was the replacement in the amended protocol of the original protocol text documented?]
   At least one of the following CV risk factors:
   a) previous MI (5 days prior to randomization);
   b) previous stroke (5 days prior to randomization);
   c) previous coronary, carotid or peripheral arterial revascularization;
   d) angina with documented ischemic changes (at least 2 mm ST segment depression on ECG during a Graded Exercise Test [GXT]; or with a cardiac imaging study positive for ischemia); or unstable angina with documented ischemic changes (either ST segment depression of at least 1 mm or an increase in troponin above the normal range but below the range diagnostic for acute MI);
   e) microalbuminuria or clinical proteinuria (an albumin:creatinine ratio ≧30 μg/mg in at least one first morning urine sample or timed collection of urine with albumin excretion ≧20 μg/min or ≧30 mg/24 hours or total protein excretion ≧500 mg/24 hours);
   f) left ventricular hypertrophy by electrocardiogram or echocardiogram;
   g) significant stenosis on angiography of coronary, carotid, or lower extremity arteries (ie, 50% or more stenosis);
   h) ankle-brachial index (ABI)<0.9.
4. Provision of signed and dated informed consent prior to any study procedures.
5. Ability and willingness to complete study diaries and questionnaires.
6. Demonstrated ability to use the self-glucose-monitoring device, and to self-inject insulin prior to randomization.
7. A negative pregnancy test for all women of childbearing potential (ie, ovulating, pre-menopausal, and not surgically sterile) and the agreement of these women to use a reliable method of birth control to prevent pregnancy during the duration of the study.
8. Willingness to discontinue prior omega-3 PUFA supplements for the duration of the study.

### Example 3

### Selection of Study Population—Exclusion Criteria

People with any of the following characteristics will be excluded from the study:
1. Type 1 diabetes.
2. Requiring ambulatory insulin treatment or uncontrolled or symptomatic hyperglycemia that is likely to require the addition of ambulatory insulin therapy or a new antidiabetic agent either before or within 2 weeks after randomization.
3. Known anti-glutamic acid decarboxylase antibody (anti-GAD Ab) positivity in the past.
4. Screening glycated hemoglobin ≧150% of the ULN for the laboratory (eg, ≧9% if the ULN is 6%).
5. Unwillingness to inject insulin or perform self-monitoring of BG.
6. Nonadherence to the run-in requirement to inject placebo insulin and do capillary glucose monitoring for at least 4 days prior to randomization.
7. Currently planned coronary artery bypass grafting (CABG) or CABG within the 4 years prior to screening—however, patients with angina, MI, or stroke since a previous CABG will be eligible for randomization, even if the last CABG was within 4 years.
8. Serum creatinine >2.0 mg/dL (176 μmol/L) at screening.
9. Active liver disease, or alanine aminotransferase (ALT) or aspartate aminotransferase (AST) >2.5 times ULN at screening.
10. Chronic or recurrent treatment with systemic corticosteroids, or niacin treatment for hyperlipidemia.
11. Heart failure of NYHA Functional Class III or IV.
12. Expected survival of <3 years for non-CV causes such as cancer.
13. Any other factor likely to limit protocol compliance or reporting of adverse events (AEs).
14. Unwilling or unable to discontinue TZDs.
15. Simultaneous participation in any other clinical trial of an active pharmacologic agent.
16. Unwillingness to permit sites to contact their primary physicians to communicate information about the study and the participant's data and treatment assignment.
17. History of hypersensitivity to the investigational products.
18. Previous randomization in this study.
19. A prior heart transplant, or awaiting a heart transplant.
20. Known infection with human immunodeficiency virus (HIV)

### Example 4

### Study Treatments

### Investigational Medicinal Products

### Insulin Glargine

Patients randomized to insulin glargine received injections of insulin glargine 100 U/mL solution (Lantus®) with a pen device (Optipen®) QD SC in a titrated regimen targeting an FPG level of ≧95 mg/dL (5.3 mmol/L) according to suggested algorithms. Treatment continued until a prespecified number of patients had experienced at least one component of the primary outcome (2 200 first coprimary outcomes);

### Ethyl Esters of Omega-3 PUFA

Patients randomized to omega-3 PUFA were to receive one gelatin capsule of ethylesters of omega-3 PUFA (icosapent ethyl esters 465 mg and doconexent ethyl esters 375 mg; Omacor®) QD per os (PO). As with insulin glargine therapy, treatment was to continue until a prespecified number of patients had experienced at least one component of the primary outcome.

### Reference Therapy

### Standard Care

Standard care was the reference therapy for insulin glargine.

Diabetic patients (and patients who developed diabetes after randomization) who were randomized to receive standard care were treated according to current (at that time) guidelines and the best judgment of the treating physician. Standard care did not include glucose-lowering drugs for nondiabetic patients. Insulin was not to be used in the standard care group until a patient had been taking maximal doses of treatments from at least 2 of the following different classes of oral glucose-lowering agents:
SU or MGT;
metformin (MET) or another biguanide;
TZD.

For patients taking less than maximal doses of at least 2 of these classes of OAD, the Investigator was to consider increasing both oral agents to maximal dose, or adding an oral agent from a third class, before beginning insulin. If the Investigator chose to add insulin before this, he or she was required to complete a report justifying the use of insulin. Whenever insulin was added, the Investigator or physician could reduce or stop some or all of the OADs at his/her discretion.

### Placebo

Placebo was the reference therapy for omega-3 PUFA.

Patients randomized to the omega-3 PUFA placebo received one matching gelatin capsule containing olive oil QD PO.

### Dosage Schedule

Insulin glargine doses were adjusted according to both laboratory and capillary plasma glucose results.

### Treatment Assignments

Randomization was stratified by investigational site.

Participants were randomized using a centralized telephone randomization system. Each randomized participant was assigned a unique number, which was used throughout the study.

### Blinding, Packaging, and Labeling

The investigational products (insulin glargine, placebo saline for run-in injection) has been packaged by Sanofi. Ancillary medication (metformin, SU) has been obtained through local pharmacies.

The comparison of insulin glargine to standard dysglycemia treatment has been carried out in open-label fashion.

### Example 5

### Summary of Performance of the Origin Study

### Methods

ORIGIN was an international randomized factorial trial of the effect of titrated basal insulin therapy versus standard care and of omega 3 fasty acid supplements versus placebo on incident CV outcomes. Results of the omega 3 fatty acid arm are reported separately (REF). Participants age 50 or older with a prior CV event (myocardial infarction, stroke, or revascularization procedure); angina with documented ischaemia; albuminuria; left ventricular hypertrophy; angiographic evidence of ≧50% stenosis of a coronary, carotid, or lower extremity artery; or an ankle/brachial index <0.9 were recruited if they also had a history of type 2 diabetes that was stable on 0 or 1 oral agent; or IFG, IGT or newly detected diabetes based on either a FPG ≧6.1 mmol/L [110 mg/dL] or a 2 hour plasma glucose ≧7.8 mmol/L [140 mg/dL] after a 75 g oral glucose load. The HbA1c level of people with prior diabetes had to be low enough to minimize the likelihood that insulin would be needed to maintain glycemic control during follow-up if allocated to standard care. Key exclusion criteria included unwillingess or an inability to inject insulin or do capillary glucose testing, a clear indication for, or intolerance to insulin or omega 3 fatty acids, unwillingness to stop thiazolidinediones if allocated to glargine, heart failure, or coronary artery bypass surgery within the prior 4 years with no intervening CV event. The study was approved by each site's ethics committee and all participants provided written informed consent.

### Interventions and Follow-Up Schedule

Participants were asked to self-administer daily subcutaneous saline injections and to check their capillary glucose levels during a 10 day run-in period. Adherent participants were then provided with lifestyle advice and randomly allocated to either insulin glargine (Lantus™) or standard approaches to glycaemic control. Participants allocated to insulin glargine who were also taking a thiazolidinedione stopped that medication at the time of randomization; otherwise the insulin glargine was added to their glycemic regimen. These participants were instructed to inject insulin glargine in the evening, starting at 2, 4 or 6 units (depending on their initial FPG) and to increase the dose at least once per week targeting a self-measured FPG level ≦5.3 mmol/l (95 mg/dl) and ≧4 mmol/l (72 mg/dl). If target FPG levels could not be achieved without symptomatic hypoglycemia, investigators were permitted to replace glyburide used at baseline with a comparable dose of glimepiride; to reduce or stop all other glucose-lowering drugs; and/or to add metformin. If participants developed uncontrolled hyperglycemia, investigators were permitted to add rapid-acting insulin. No other glucose-lowering medication could be added or increased. FPG levels were measured in the local laboratory at every visit and the results were regularly reviewed along with the dose of insulin to ensure that insulin was being effectively trirated. People not diagnosed with diabetes by the time of the penultimate study visit down-titrated insulin glargine by 10 units per day and stopped any metformin that was prescribed. If glucose levels remained in the nondiabetic range, they were scheduled for a 75 g oral glucose tolerance test 3-4 weeks later; if this test did not diagnose diabetes, it was repeated 10-12 weeks later.

Participants allocated to standard care continued the glucose-lowering therapy that they were taking before randomization. Anyone who had diabetes at baseline or who developed it during the trial was instructed to self-monitor glucose levels. Investigators were advised to manage glycemia using standard approaches according to their best judgment based on the clinical status and clinical practice guidelines, and were permitted to add, increase, reduce or stop any glucose-lowering drug except insulin glargine. Only metformin and sulfonylureas were provided by the study if required. FPG levels were measured in the local laboratory annually for people without diabetes and at 2 years and study end for people with diabetes. People without a diagnosis of diabetes by the last study visit were scheduled for a 75 g oral glucose tolerance test 3-4 weeks later; if this test did not diagnose diabetes, it was repeated 10-14 weeks later.

Outcomes and other data were collected at scheduled study visits at 0.5, 1, 2, and 4 months after randomization and every 4 months thereafter. Weight, waist and hip circumference were measured annually. HbA1c levels were assayed in local laboratories at every visit for the first year and then annually in people without diabetes and every 4 months for people with diabetes. A first morning urine collection was sent centrally and assayed for creatinine and albumin at baseline, 2 yrs and study end.

### Outcomes

There were 2 co-primary composite CV outcomes. The first was CV death, non-fatal MI or non-fatal stroke, and the second was a composite of any of these events or a revascularization procedure or hospitalization for heart failure. Secondary outcomes included a composite microvascular outcome comprising a doubling of serum creatinine from baseline, progression of albuminuria category from normoalbuminuria or microalbuminuria to microalbuminuria or overt nephropathy, renal replacement therapy, renal death, retinal photocoagulation or vitrectomy for retinopathy. They also included new type 2 diabetes developing by the time of the 1^{st} post-trial oral glucose tolerance test in participants without baseline diabetes, and all-cause mortality. Other outcomes included incident cancers, CV hospitalizations, and angina. Cardiovascular and cancer outcomes were reviewed by adjudicators masked to treatment allocation. Episodes of hypoglycaemia since the prior visit were recorded at each visit. Symptomatic hypoglycemia was classified as confirmed if a concomitant recorded capillary glucose level was <3 mmol/L (54 mg/dL). Severe hypoglycemia was defined as hypoglycemia that required assistance plus either prompt recovery with glucose or glucagon or a documented capillary glucose ≦2.0 (36 mg/dL). New diabetes was diagnosed if 2 consecutive FPG levels within a 4-month period were ≧7 mM (126 mg/dL) during the trial; a diagnosis of diabetes was made by a physician and a pharmacologic antidiabetic agent was being taken and there was evidence of either a FPG ≧7 mM (126 mg/dL) or any glucose value ≧11.1 mM (200 mg/dL); either 1 or more capillary glucose levels were ≧11.1 mM (200 mg/dl) and a lab-measured FPG was ≧7 mmol/l (126 mg/dl) or a lab measured random glucose level was ≧11.1 mM (200 mg/dl) during down-titration of glargine insulin; or any FPG was ≧7 mM (126 mg/dl) or 2 hour plasma glucose was ≧11.1 mM (200 mg/dl) during the first oral glucose tolerance test.

### Trial Conduct

A mean follow-up period of approximately 4 years was originally planned. This was extended by 10 months before recruitment had been completed after it became clear that it took approximately 8 months of insulin glargine self-titration to achieve a median FPG ≦5.3 mmol/l. Subsequently, in light of clinical trials published in 2008 suggesting that a longer duration of follow-up may be required to detect any effect of a gluco-metabolic intervention, and without any knowledge of treatment effects, the Steering Committee extended the trial for 2 more years.

Insulin glargine (Lantus®) was provided by Sanofi and omega-3-acid ethyl esters 90 (Omacor®) and placebo were provided by Pronova Biocare AS. Study data were collected and independently analyzed by the ORIGIN Project Office based at the Population Health Research Institute (PHRI) in Hamilton, Ontario, Canada.

### Statistical Analyses and Power

Data were analyzed using SAS (version 9.1 for Solarus) according to an intention-to-treat approach described in the protocol and a predefined statistical analysis plan. Participants who were lost to follow-up, formally withdrew or did not consent to either of the protocol extensions were censored at the time of their last contact. Baseline characteristics were summarized using means and standard deviatons, medians and interquartile ranges, or counts and percentages as appropriate. Time-to-event curves were constructed using product limit estimation and compared using stratified log-rank tests. Hazard ratios for each outcome were calculated using Cox regression models adjusted for the factorial allocation, baseline diabetes status and a history of a prior CV event before randomization as described in the protocol. The proportional hazards assumption was assessed by testing for the interaction of time with treatment group. The incidence of new diabetes in each allocated group between randomization and the first post-study oral glucose tolerance test was compared using a Cochran-Mantel-Haenzel test stratified by factorial allocation and a prior CV event, and an odds ratio was calculated; durability of this effect was assessed by repeating the analysis after the 2^{nd} post-study oral glucose tolerance test.

This overall type I error of 5% for the two co-primary outcomes was partitioned such that the first co-primary outcome was tested at P=0.044 and the second co-primary outcome was tested at P=0.01; the non-additivity of these error rates reflects the correlation between these co-primary outcomes. The nominal level of significance for all other analyses was P=0.05. Predefined subgroups were sex, age (<65 or ≧65), geographical region; ethnicity, baseline diabetes status; body mass index ≦30 or >30 kg/m²), prior CV event, and factorial allocation.

Based on an annual incidence of the first coprimary outcome of 2.8%, a mean follow-up of 6.5 years, a type 1 error rate of 0.044, noncompliance with insulin of 20% in the glargine group and use of insulin in the control group of 5%, and a 12 month delay before an effect of the intervention emerges, it was estimated that 12,500 participants would yield 2200 first coprimary outcomes and 3900 second coprimary outcomes and provide 90% power to detect relative risk reductions of 18% and 16% respectively.

### Example 6

### Results

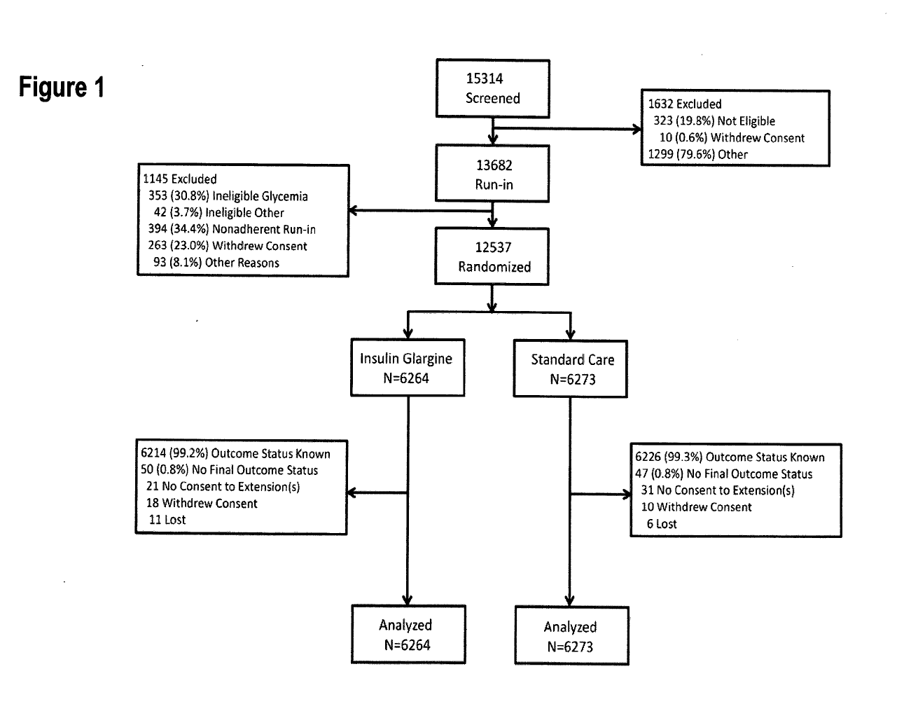

12,537 participants (mean age 63.5 yrs; 35% female) were enrolled from 573 clinical sites in 40 countries. Participants were randomized to either insulin glargine or standard care between September 2003 and December 2005 and followed for a median (IQR) period of 6.2 (5.8, 6.6) years. At study end the primary outcome status was known for 12443 (99.3%) participants (FIG. 1). Approximately 82% of participants had a prior history of diabetes of mean (SD) duration 5.4 (6.0) years, 6% had newly detected diabetes and 12% had IFG and/or IGT. The median (IQR) FPG at baseline was 6.9 (6.1, 8.2) mmol/l. 5052 (40%) participants were not taking diabetes drugs, 3435 (27%) were on metformin and 3711 (30%) were on a sulfonylurea. These and other key baseline characteristics of the 2 treatment groups are shown in Table 1. Of note, data from an additional 75 individuals in 3 sites located were excluded (before the trial was closed or unblinded) at the request of their respective national regulatory agencies following site audits.

50% of participants allocated to the addition of insulin glargine to their regimen achieved a FPG level ≦5.2 mmol/l by 1 year that was maintained throughout the trial (Table 2). The median insulin dose taken to maintain this degree of glycemic control rose from 0.28 U/kg at year 1, to 0.40 U/kg by year 6. At the time of the penultimate visit (i.e. before insulin glargine was tapered and discontinued in people with no diagnosis of diabetes) insulin glargine had been permanently discontinued by 17% of insulin glargine group participants (Table 5). By this time, 35% were on no oral agents, 47% were taking metformin, and 14% were taking ≧2 oral agents (Table 3).

Few standard care group participants used insulin during the trial (Table 2). Thus at 2 years only 208 (3.5%) standard care participants were using any insulin and at the 5 year visit only 494 (9.0%) were using any insulin. By study end, 19% were on no oral agents, 60% were taking metformin, and 42% were taking ≧2 oral agents (Table 3). In addition to the large contrast in insulin use, the 2 different therapeutic approaches achieved a 1.6 mmol/l (29 mg/dl) difference in FPG by 2 years and approximately a 0.3% difference in A1C levels during the trial (Table 2).

The incidence of the first episode of severe hypoglycemia was 1.00 per 100 person-years in the insulin glargine group and 0.31 per 100 person-years in the standard care group (P<0.001). The incidence of the first episode of nonsevere symptomatic hypoglycemia that was confirmed by a self-measured glucose level 3 mmol/l (54 mg/dl) was 9.81 and 2.68 per 100 person-years in the insulin glargine and standard care groups (P<0.001) respectively, and the incidence of the first episode of any (i.e. confirmed or unconfirmed) hypoglycemia was 16.73 and 5.16 per 100 person-years in the 2 groups respectively. A total of 2691 (43%) insulin glargine participants and 4694 (75%) standard care participants did not experience any episode of symptomatic hypoglycemia during the entire trial (Table 4). Insulin glargine group participants gained a mean of 1.6 kg whereas standard care participants lost a mean of 0.7 kg.

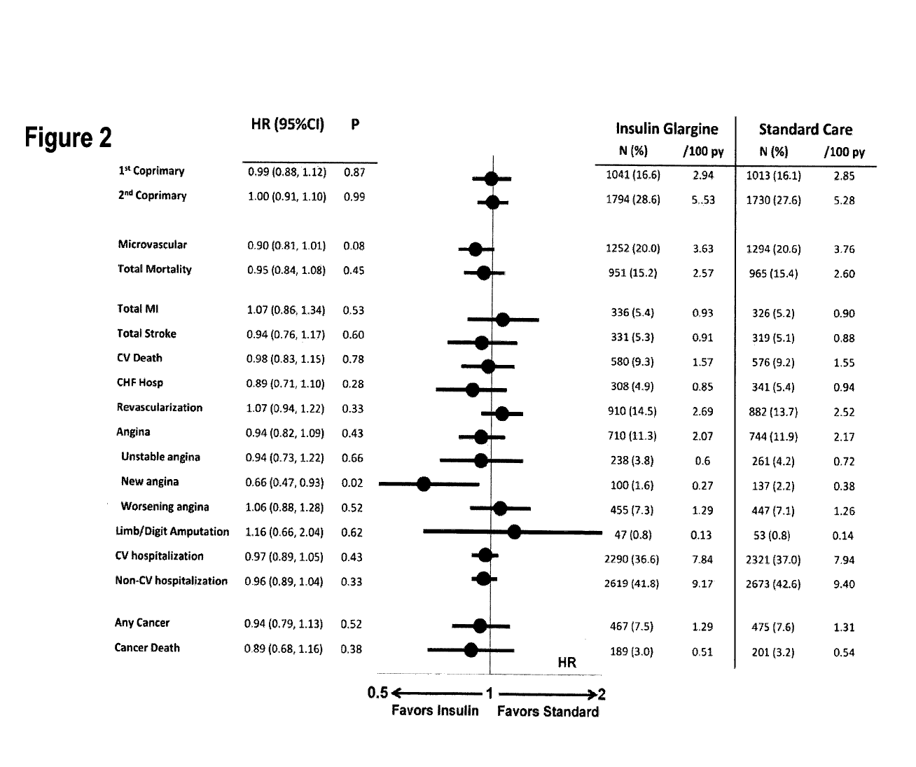

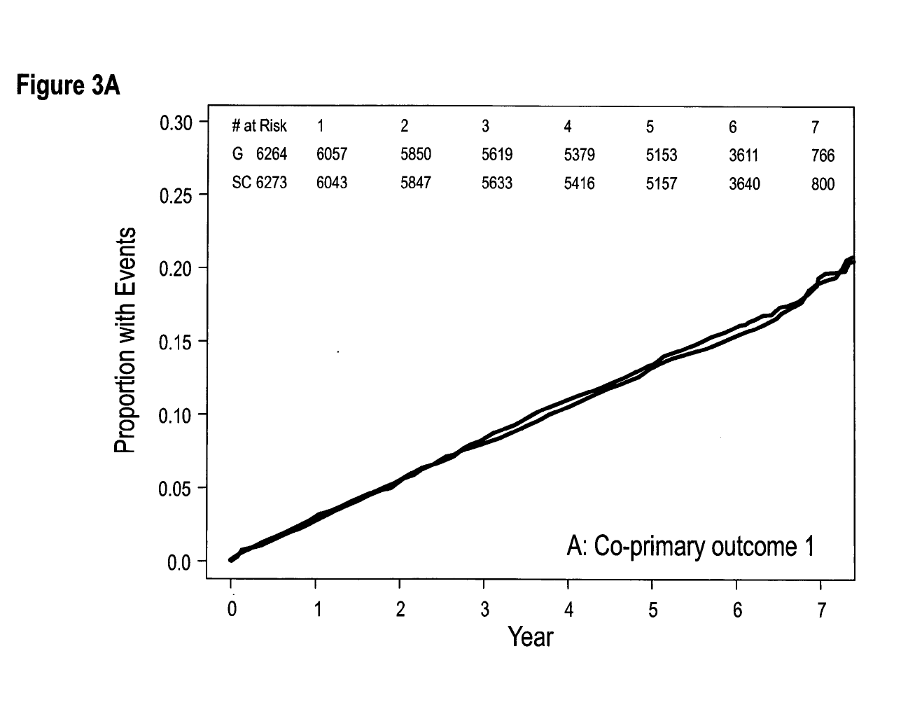

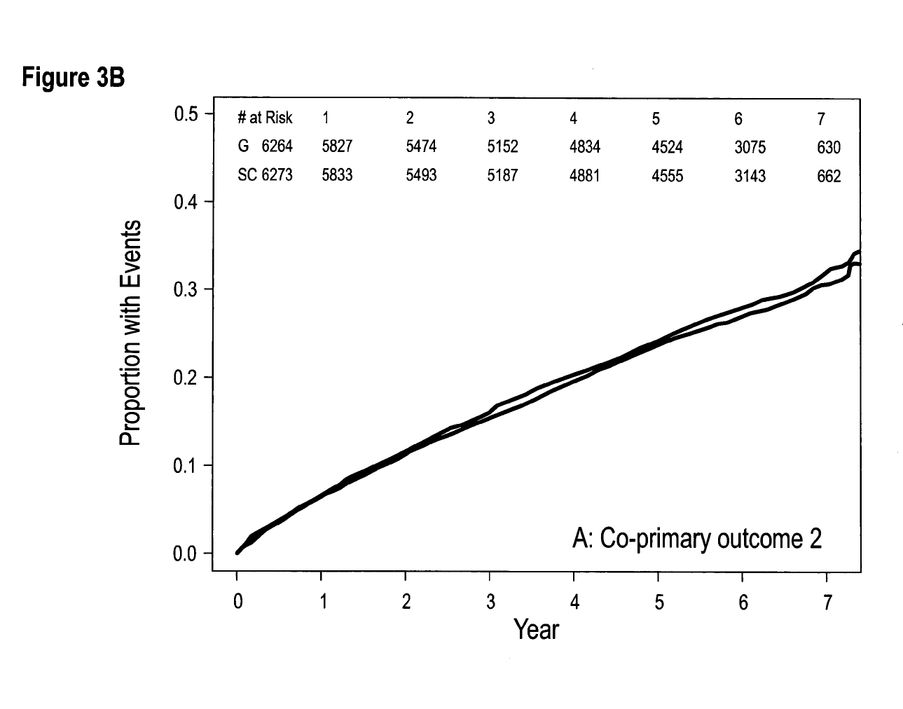

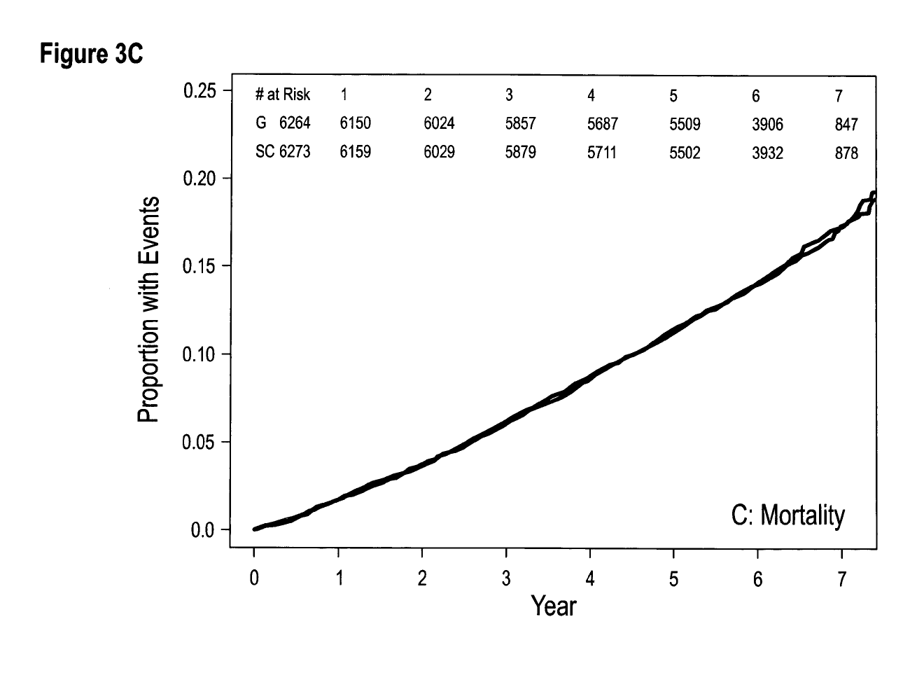

There was no statistical evidence for an interaction between the effects of insulin glargine and the omega 3 fatty acid trial for any of the outcomes (P>0.15 for all outcomes). The incidence of both co-primary outcomes did not differ between treatment groups (FIGS. 2 and 3). Specifically the incidence of the composite outcome of nonfatal MI, nonfatal stroke or CV death (i.e. the first co-primary outcome) was 2.94/100 person-years and 2.85/100 person-years for the insulin glargine and standard care group respectively (adjusted HR 0.99: 95% CI 0.88, 1.12; P=0.9). For the second co-primary outcome the incidence was 5.53/100 person-years and 5.28/100 person-years for each group respectively (adjusted HR 1.00: 95% CI 0.91, 1.10; P=0.99). The effect of the intervention on the 2 co-primary outcomes was similar across key subgroups (Supplementary FIG. 1). Of note was statistical evidence of variation by geographic region for the 1^{st} co-primary outcome (interaction P=0.005) that was not evident for the larger 2^{nd} co-primary outcomes (interaction P=0.09) or for ethnicity with either outcome.

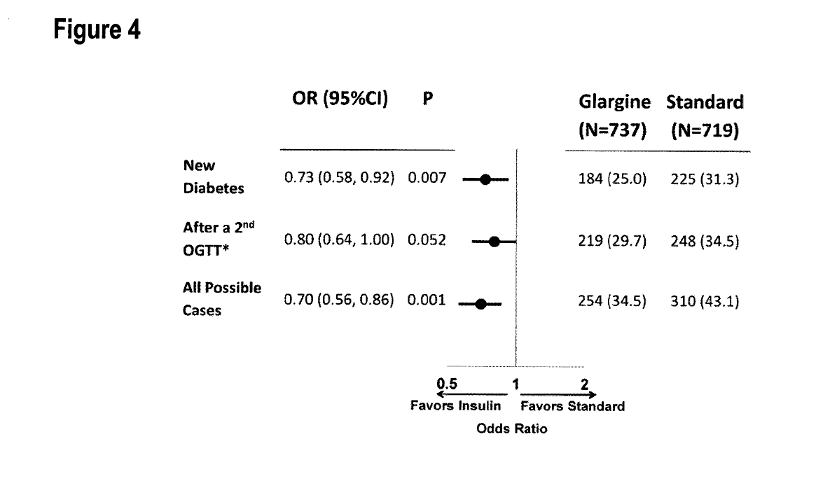

There was also no statistically significant difference in mortality or microvascular outcomes, although there is a trend that treatment with insulin glargine is beneficial with regard to microvascular outcomes. Surprisingly, participants without diabetes at randomization who were allocated to insulin glargine were 27% less likely (FIG. 4) to develop protocol-defined diabetes than standard care participants (i.e. 25% versus 31%: OR 0.73, 95% CI 0.58, 0.92; P=0.007). When individuals without diabetes based on the the 1^{st} oral glucose tolerance test had it repeated a median of 100 (94-112) days after insulin was stopped, additional cases of diabetes were detected in both groups so that the total the rates were 30 and 35% respectively (OR 0.80, 95% CI 0.64, 1.00; P=0.052). Under an early intervention with insulin glargine a highly significant effect on the development of new angina was detected. In the glargine group 100 patients developed a new angina (1.6%) whereas in the standard care group 137 (2.2%) developed a new angina. This significant difference (p=0.02) was observed after the 6-7 years exposure to the different regimens.

Finally when cases of diabetes that were suspected of having developed during the trial (but that did not meet all of the predefined criteria) were also included the incidence of new diabetes was reduced by 30% (i.e. 35% versus 43%: OR0.70, 95% 010.56, 0.86; P=0.001). There was no difference in the incidence of any cancer or cancer death (FIG. 2).

### Example 7

### Microvascular Outcomes

There was a significant reduction in clinical microvascular events. This includes clinical events like laser surgery, renal failure, blindness, end-stage renal disease, or renal death. Supporting this last, there was a significant reduction in laser surgery or vitrectomy for diabetic retinopathy. There was also a strong trend to reducing doubling of baseline serum creatinine. The results are summarized in Table 6.

Furthermore, the data obtained support an effect on microvascular disease progression in the subgroups of patients having a higher baseline A1c, and atrial fibrillation.

Patients with baseline A1c<6.4% had a risk reduction (RR) (glargine: subcutaneous) of 1.08 (not significant), patients with A1c≧6.4%, RR=0.88 (0.79-0.98), thus statistically significant because the confidence intervall excluded 1.

Patients with a history of atrial fibrillation at baseline had a RR of 0.74 (0.55-0.98), and a RR for Clinical Microvascular outcomes (non-laboratory-based) of 0.42 (0.19-0.91).

The microvascular outcome was a composite of: laser surgery or vitrectomy or blindness for diabetic retinopathy; development of renal death or the need for renal replacement treatment (dialysis or transplantation); doubling of serum creatinine; or progression from lesser to greater severity of microalbuminuria. The last 2 components are laboratory-based—the others are “clinical” microvascular outcomes.

### Example 8

### Cognition Outcomes

Overall, there was a strong trend (p=0.075) for glargine treatment to be associated with fewer impaired patients. The data, as summarized in Table 7, reflect the Mini-Mental Status Exam (MMSE) [61] data for the number of participants scoring 24 or less at various timepoints (mild impairment). These patients were examined because they represent those at greater risk of further deterioration during the study, and still with enough patients to confer adequate power to make statistical comparisons. There was a significant reduction of cases of mild impairment from baseline at about 4 years.

### Example 9

### Glargine Leads to a Significant Lowering of Triglyceride Concentration in the Blood

By the ORIGIN study it has been shown that the triglyceride concentration in the blood decreased in a statistically significant manner for patients treated with glargine vs. standard care: −0.21 (0.03) [glargine] vs. −0.15 (0.03) [standard care] (P<0.001).

### Example 10

### Glargine Leads to a Significant Lowering of Cholesterol Concentration in the Blood

By the ORIGIN study it has been shown that the triglyceride concentration in the blood decreased in a statistically significant manner for patients treated with glargine vs. standard care:

Total cholesterol change from baseline to end-of-study (in mmol/L):

| Glargine | Standard Care | p |
|---|---|---|
| −0.41 | −0.37 | 0.044 |

### Example 11

### Attainment and Maintenance of A1C<6.5 or <7.0% with Titrated Basal Insulin or Standard Oral Therapy in the ORIGIN Trial—Detailed Results

OBJECTIVE—To assess the success and baseline predictors of maintaining glycemic control for up to 5 years of therapy using basal insulin glargine versus standard glycemic care in people with dysglycemia treated with 0 or 1 oral glucose-lowering agents.
RESEARCH DESIGN AND METHODS—Data from 12, 537 participants in the ORIGIN trial were examined by baseline glycemic status (with or without type 2 diabetes) and by therapeutic approach (titrated insulin glargine or standard therapy) using an intention-to-treat analysis. Median values for FPG and A1C during randomized treatment and percentages attaining and maintaining <6.5% or <7.0% A1C were calculated. Factors independently associated with success in reaching these levels of control were analyzed with linear regression models.
RESULTS—Both treatment strategies kept median FPG and A1C at or below baseline values, which were 6.9 mmol/l (125 mg/dl) and 6.4% respectively. Absence of diabetes and lower baseline A1C, independent of each other, were associated with greater likelihood of maintaining 5-year mean A1C<6.5%. Allocation to basal insulin glargine was also a strong predictor of maintaining A1C<6.5% (OR 2.98, 95% CI 2.67, 3.31; p<0.001) after adjustment for other independent predictors. This effect was noted overall and within all of the analyzed subgroups.
CONCLUSIONS—Intervention early in the natural history of dysglycemia can prevent worsening of control for at least 5 years. Maintaining A1C<6.5% is especially likely when A1C is lower at baseline and when basal insulin is used.

There is a strong relationship between hyperglycemia and micro- and macrovascular complications of type 2 diabetes (1-4), and treatment studies have verified that improved glycemic control can limit some of these complications (5-8). However, diabetes is a progressive disorder and treatment often does not prevent a gradual increase of hyperglycemia over time (9-11). The Outcome Reduction with an Initial Glargine Intervention (ORIGIN) trial compared the medical outcomes of two treatment methods designed to maintain nearly normal glycemic control early in the natural history of dysglycemia, including both individuals with elevated glucose levels not meeting the criteria for diabetes and people with diabetes with limited prior therapy (12). The 12,537 participants were randomized to treatment with either basal insulin glargine, which was systematically titrated to maintain fasting plasma glucose ≦5.3 mmol/l (95 mg/dl), or to standard therapy. The cardiovascular and other medical outcomes of ORIGIN have been reported previously (13,14). Here we report the ability of each regimen to keep HbA1c (A1C) below guideline-recommended target levels for up to 5 years of follow-up, as well as the baseline characteristics of participants associated with achieving this goal.

### Research Design and Methods

### Participants

The rationale and design of the ORIGIN trial were reported previously (9). In brief, it was a multinational randomized trial with a 2×2 factorial design which tested two pairs of interventions. Titrated basal insulin glargine was compared with standard stepwise oral therapy, and an omega-3 fatty acid supplement with placebo. Participants were required to have a prior cardiovascular event or other evidence of high cardiovascular risk together with documented dysglycemia, defined as either impaired fasting glucose, impaired glucose tolerance (or the two together), or newly detected or previously diagnosed type 2 diabetes. Participants with diabetes could be treated with lifestyle alone or accompanied by no more than a single oral glucose lowering agent. The present analysis concerns the glycemic intervention, with use of omega-3 fatty acids included only as a covariate. Data from a population of 12,537 individuals in 40 countries were assessed.

### Interventions

Participants assigned to standard therapy continued their prior oral therapies and were managed according to the investigators' judgement and local guidelines for glycemic control and therapeutic approaches. Investigators were advised not to prescribe insulin for standard participants unless they were on full doses of 2 or more oral agents. If insulin was added glargine was not to be used. Participants assigned to basal insulin glargine who were taking a thiazolidinedione prior to randomization stopped this medication but continued to take other glucose-lowering agents. Insulin glargine (Lantus®, Sanofi) was added to their regimen starting at 2 to 6 units daily, based on fasting glucose levels. Participants were advised to inject the insulin in the evening and to self-titrate the dosage using a simple algorithm supported by the site investigators. Self-measured, plasma-referenced fasting capillary blood glucose tests were done at least twice-weekly to guide titration, with the goal of achieving and maintaining fasting glucose 5.3 mmol/l (≦95 mg/dl). Other oral agents could be continued, reduced, or discontinued as judged appropriate during treatment with insulin glargine. The only oral agent that could be added (if not previously used) was metformin, which the site investigator initiated for individual participants at doses of 500-1000 mg/day if judged necessary to limit the risk of hypoglycemia. The importance of lifestyle management was continuously reinforced in both treatment groups.

### Measurements

In addition to self-measured glucose tests, venous blood for measurement of fasting plasma glucose (FPG) and A1C at local laboratories was collected at intervals during treatment. In the case of A1C, measurements were done at baseline, yearly thereafter, and at the end of treatment for all participants. Measurements of FPG were done annually and at the end of treatment for all participants in the glargine treatment group, and at baseline, after 2 years, and at the end of treatment for those using standard therapy.

### Statistical Analysis

Summary statistics were computed for baseline characteristics of the whole population and for subgroups by glycemic treatment allocation and by glycemic status at enrollment (dysglycemia without diabetes, or diabetes). Median FPG and A1C with inter-quartile ranges were computed for each subgroup for all time points. Percentages of participants in each subgroup having A1C<6.5% and <7.0% (two levels commonly identified as targets for glycemic control [15,16]) at each time-point were calculated. To determine associations of baseline characteristics, glycemic status, and treatment allocation with glycemic outcomes, findings for all randomized participants up to 5 years of treatment were analyzed using statistical models. Data after 5 years of treatment were not included because many participants did not have follow-up beyond that interval due to the timing of randomization. Attainment of A1C<6.5% or <7.0% was defined as having values below those levels at 1 year; maintenance of A1C during treatment was defined as having the mean of all values from 1 year to the last available measurement up to 5 years at those levels. All analysis of the relationships between baseline characteristics and glycemic control levels were performed using linear regression models. Characteristics with a univariable p<0.1 in univariate analyses were entered into multivariable models. The independent effect of allocation to basal insulin glargine versus standard treatment was assessed by adding allocation to a final multivariable model which included all variables statistically significant at p<0.05 in these multivariable models. The unadjusted effect of allocation to insulin glargine was estimated using logistic regression and statistical tests for interactions between allocation and these subgroups were calculated and displayed as a forest plot.

### Results

### Baseline Characteristics

The characteristics of the ORIGIN population at enrollment, divided by treatment assignment and glycemic status, are shown in Table 8. Of 12,537 randomized, 6,264 were assigned to treatment with insulin glargine and 6,273 to standard care. The two randomized treatment groups were alike in baseline characteristics. Eighty-eight percent of participants had either a prior diagnosis of diabetes (of mean duration 5.4 years) or newly detected diabetes. The 12% without diabetes clearly differed from those with diabetes in FPG and A1C levels and also in other ways, including more frequent prior CV events, use of alcohol, depression, and use of statins and beta blockers. For the whole population, the mean age was 63.5 years, median FPG 6.9 mmol/l, and median A1C 6.4%.

### Glycemic Responses During Randomized Treatment

### Median FPG

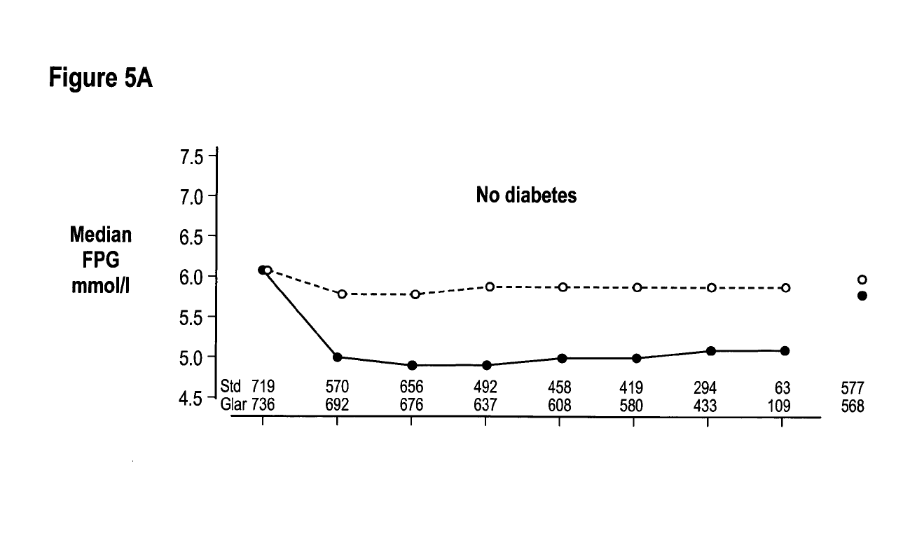

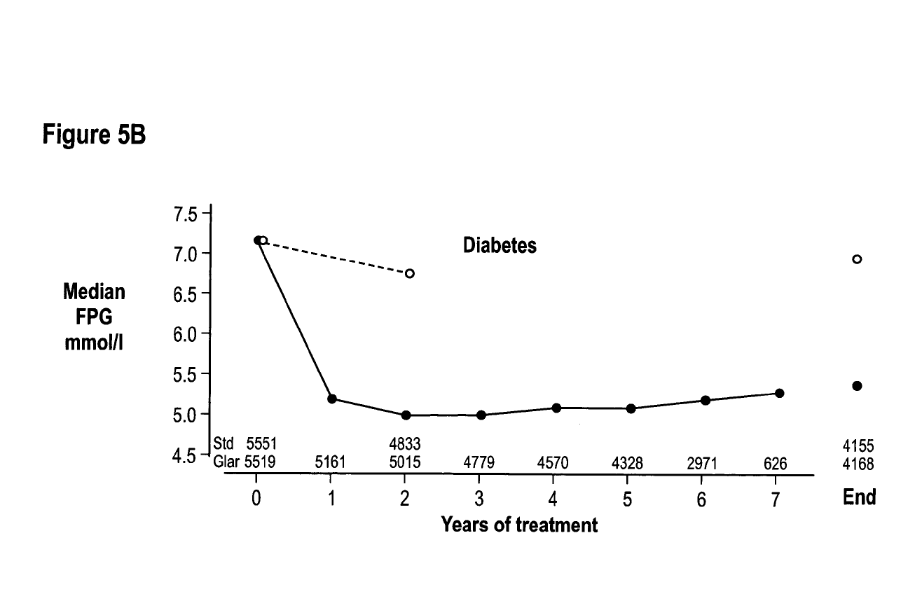

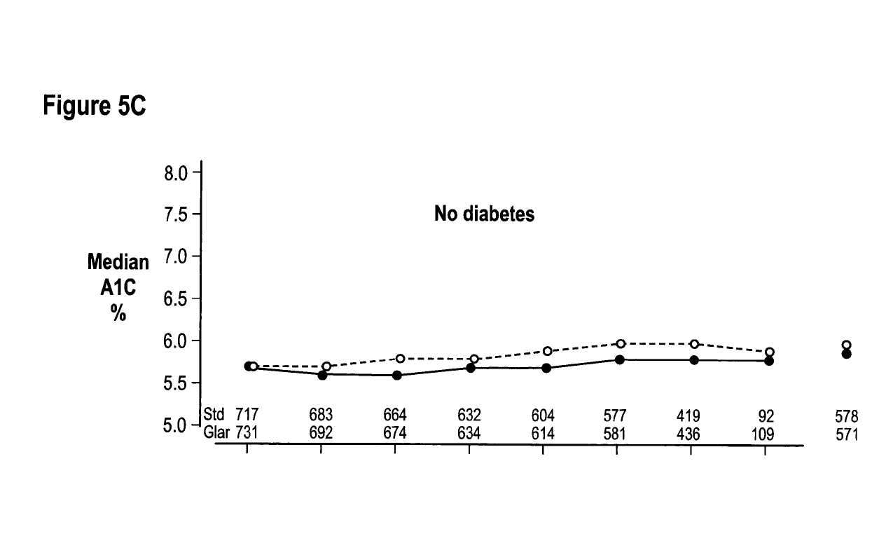

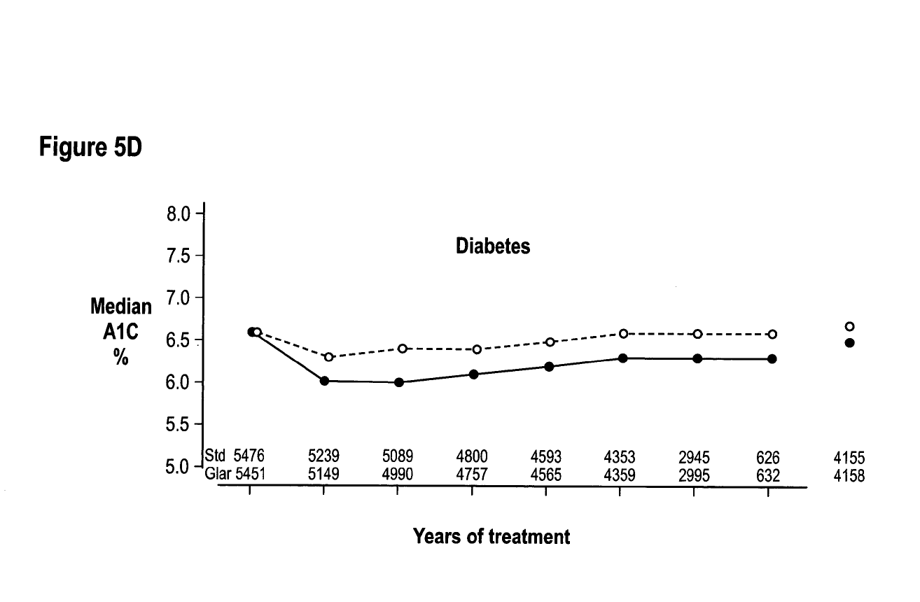

The median period of follow-up on randomized treatment was 6.2 years. The effect of treatment allocation on the responses of FPG and A1C during treatment is shown in FIG. 5. For participants without diabetes the median FPG (inter-quartile range) was 6.1 (5.5-6.4) mmol/l prior to randomized treatment (FIG. 5A). Standard care led to little change of FPG in this subgroup, but insulin glargine caused a sustained decrease to median values of 5.0 (4.5-5.5), 4.9 (4.5-5.5), 5.0 (4.5-5.7), and 5.1 (4.5-5.8) mmol/l at 1, 2, 5, and 7 years. For participants with diabetes the median baseline FPG was 7.2 (6.2-8.4) mmol/l. With standard care the values at 2 years and the end of treatment were 6.8 (5.9-8.1) and 7.0 mmol/l (5.9-8.4) mmol/l (FIG. 5B). Treatment with glargine reduced median FPG to 5.2 (4.6-5.9), 5.0 (4.4-5.8), 5.1 (4.5-6.1), and 5.3 (4.5-6.4) mmol/l after 1, 2, 5, and 7 years.

### Median A1C

For participants without diabetes A1C changed little from baseline with either regimen (FIG. 5C). With standard therapy the median A1C was 5.7 (5.4-6.1) % at baseline, 5.7 (5.4-6.1) at 1 year, and 6.0 (5.6-6.4) after 5 years. For glargine-treated participants without diabetes median A1C was 5.7 (5.4-6.0) % at baseline, 5.6% (5.3-5.9) at 1 year, and 5.8% (5.4-6.1) at 5 years. For participants with diabetes the median A1C at baseline was 6.5 (6.0-7.3) (FIG. 5D). During standard care the median A1C values at 1, 2, 5 and 7 years were 6.3 (5.8-6.9), 6.4 (5.9-7.0), 6.6 (6.1-7.2), and 6.6 (6.1-7.3) %. Corresponding values during treatment with glargine declined to 6.0 (5.5-6.5), 6.0 (5.6-6.6), and 6.3 (5.8-6.9), and 6.3 (5.8-6.9) %.

### Percentages Below 7.0% and 6.5% A1C

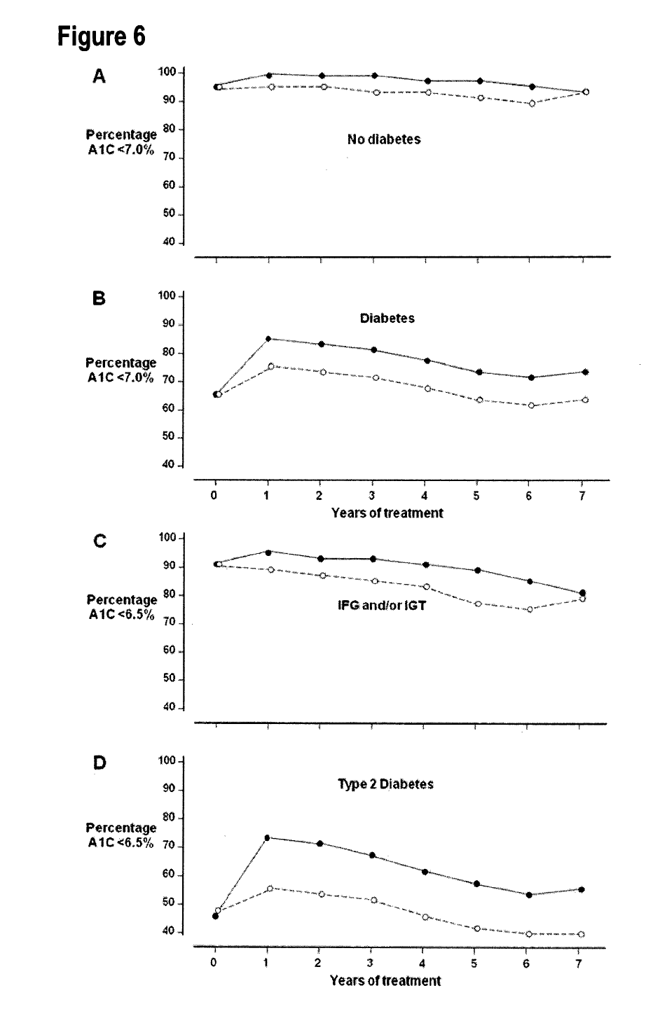

Of participants without diabetes at entry, more than 90% achieved A1C levels <7.0% and more than 75% achieved an A1C<6.5% throughout randomized treatment with both regimens (FIGS. 6A and 6C). Of participants with diabetes, 66% had an A1C<7.0% and 47% had A1C<6.5% before starting treatment (FIGS. 6B and 6D). During glargine treatment the percentages in the diabetic subgroup achieving an A1C<7.0% were 88% at 1 year and 77% at 5 years, and the percentages achieving an A1C<6.5% were 74% at 1 year and 60% after 5 years.

Multivariable models showing associations of selected baseline characteristics with attaining an A1C<6.5% or <7.0% at 1 year, and with maintaining a mean level of <6.5% or <7.0% for up to 5 years are shown in Appendix Table 1. The leading independent predictors of success based on pre-randomization characteristics were lower A1C, lack of diabetes at baseline, and reported use of alcohol. The effect of adding allocation to insulin glargine or standard care to the models is shown in Table 9. The adjusted odds ratio for success in attaining and maintaining each A1C target when using glargine compared with standard care ranged from 2.4 to 2.9 (all p<0.001). Other significant predictors of success were lower A1C, lack of diabetes, and alcohol use, whereas predictors that were significant in some but not all models included greater age, lack of a prior CV event, greater grip strength, and lower rates of albumin excretion.

### Effect of Treatment Allocation on Achieving a Mean A1C<6.5% Over 5 Years in Different Subgroups

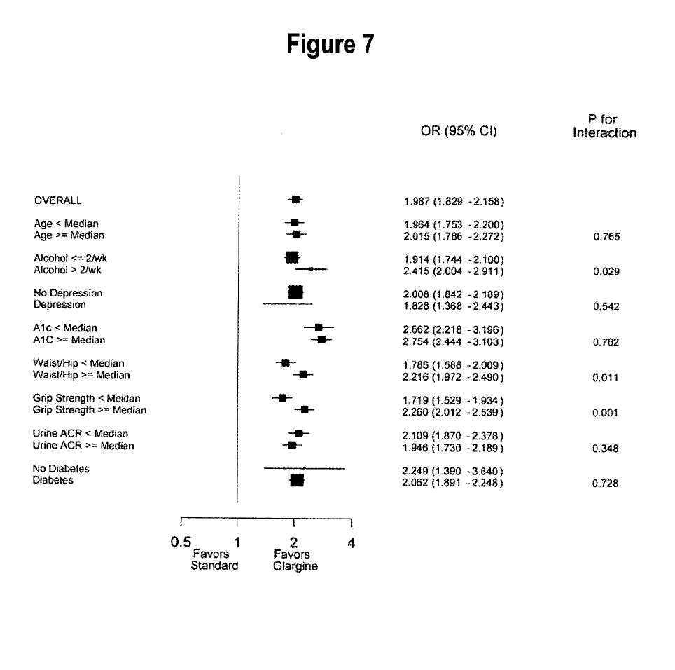

FIG. 7 shows unadjusted odds ratios for success in maintaining A1C<6.5% with glargine compared with standard therapy in baseline subgroups selected for having a significant association (p<0.05) with treatment success in the multivariable models. The insulin glargine regimen was more effective in all subgroups, with no overlap of 95% confidence intervals with unity. Two subgroups showed nominally significant interaction with treatment assignment: glargine may have been relatively more effective in participants with higher waist-hip ratios (p=0.011) and those with greater grip-strength (p<0.001).

### Glucose-Lowering Therapies

The usage of oral glucose-lowering agents prior to randomization is listed in Appendix Table 2. Less than 2% of participants with dysglycemia not meeting criteria of diabetes had used such agents prior to entry, and none at the time of oral glucose tolerance testing during screening. Of the participants with diabetes at enrollment, 32% were taking no oral therapy, 31% metformin, and 33% a sulfonylurea. Appendix Table 3 displays usage of oral agents and insulin at the end of treatment. Of the participants without diabetes at entry, 69% of those assigned to glargine and two (0.3%) of those assigned to standard care were taking insulin at the end of the study. At the end of follow-up, 21% of those randomized to glargine and 31% of those randomized to standard care were taking one or more oral agents, most often metformin (17%, 24%; p<0.003). Of the participants with diabetes at entry, insulin was used at the end by 82% of those who were assigned to glargine treatment and by 12% of those assigned to standard care (p<0.001). Oral therapies were used by 71% of participants with diabetes assigned to glargine and 88% of those assigned to standard care (p<0.001). Metformin was taken by 50% and 65% of in the glargine and standard groups, respectively, and sulfonylureas were used by 28% and 52% (each <0.001). Two or more oral agents were taken by 14% of the glargine-treated group and 42% of the standard care group.

### Hypoglycemia

The percentage of people with diabetes at enrollment having 1 or more nonsevere hypoglycemic episodes confirmed by a glucose test <3 mmol/l (<54 mg/dl) was 10.5 per 100 person-years with glargine and 3.0 per 100 person-years with standard treatment. Corresponding frequencies for those without diabetes at enrollment were 5.7 and 0.3 per 100 person-years.

### CONCLUSIONS

The methods of therapy used in ORIGIN attained and maintained excellent glycemic control of both FPG and A1C for at least 5 years of follow-up in participants with dysglycemia. With both insulin glargine and standard care the A1C levels at the end of treatment were no higher than at baseline. This pattern of glycemic control differs from that observed in some other long-term studies in which glycemic control steadily worsened over the course of 5 to 10 years (9-11). Sustained glycemic control in ORIGIN presumably resulted from the ‘treat-to-target’ schemes used in each treatment arm. The dosage of glargine was systematically adjusted seeking FPG levels 5.3 mmol/l, and metformin could be added to mitigate the risk of hypoglycemia. Similarly, during standard therapy oral medications were added and their dosage increased with the aim of keeping A1C below either 6.5% or 7.0%, depending on locally accepted guidelines. At the end of treatment 42% of those using the standard regimen were taking two or more oral agents, and 14% of participants assigned to glargine therapy were doing so. In contrast, treatment in the Belfast (9), UKPDS (10), and ADOPT (11) studies was based on assignment to monotherapy regimens, including diet alone, metformin, sulfonylurea, thiazolidinedione, or basal insulin, with escalation of therapy only under certain conditions.

The results in ORIGIN also differ from those in ADVANCE (17), ACCORD (18), and VADT (19), trials in which glucose-lowering therapies in the intensive treatment groups were systematically adjusted seeking near-normal glycemic control. In these studies the participants enrolled had longer duration of diabetes and in most cases established therapy with multiple glucose lowering agents. The A1C levels attained in ADVANCE (6.5%) and ACCORD (6.4%) were close to those at baseline in ORIGIN (6.5%), whereas those in VADT were slightly higher (6.9%). However, these values were achieved by strenuous efforts to improve control from higher levels at baseline. Hence, maintenance of A1C at or below near-normal entry levels in ORIGIN contrasts with the other trials' efforts to restore previously inadequate glycemic control. Keeping glycemic control below a level associated with increasing risk by advancing therapy as needed may be a more desirable approach than the historically common practice of allowing marked hyperglycemia to occur and then attempting to reduce levels to a lower target (20-22). This concept is in keeping with the recent adoption of A1C 6.5% as one option for timely diagnosis of diabetes to allow intervention to minimize the risk of complications (23,24).

Not surprisingly, allocation to basal insulin glargine with titration of dosage seeking normal FPG levels led to a 2-3 fold increase in the likelihood of maintaining mean A1C below 6.5% for 5 years. Moreover, this effect was observed in all of the subgroups that were examined. Other independent predictors of maintaining this level of glycemic control were the absence of diabetes, lower baseline A1C, and self-reported alcohol use. The significance of the association of more frequent use of alcohol with better glycemic responses is unclear. In contrast greater success of therapy associated with less severe hyperglycemia at baseline is consistent with other reports. Use of systematically titrated glargine was, as previously reported (13), associated with 1.6 kg gain of weight and increased of risk of hypoglycemia. However, these unwanted effects of seeking nearly normal glycemic control were less prominent in ORIGIN than in the trials in which participants had longer duration of diabetes and more elevated A1C levels at baseline (18,19). For example, the mean gain of weight with the intensive treatment regimen in the VADT was 8.2 kg (19). Also, the annual incidence of severe hypoglycemia with intensive treatment in ACCORD was 3.14% (25), whereas it was 1.00% with basal insulin and 0.31% with standard therapy in ORIGIN (13).

Limitations of the present analysis include the lack of additional information regarding the effects of the treatments used and glycemic levels attained on medical outcomes, both desirable and unwanted. Although maintenance of nearly normal glycemic control for 5 years may be predicted to delay development of complications of diabetes, the balance of risks to potential benefits remains to be determined by further analyses and additional follow-up of the participants.

In summary, intervention with basal insulin glargine or with standard care at an early stage of the natural history of dysglycemia maintained median A1C at or below the starting level for at least 5 years. Maintaining the mean of yearly A1C measurements below 6.5% was more often accomplished when the initial A1C was lower and with titrated basal insulin than with standard care.

### Legends to Tables of Example 11:

Table 8: Logistic regression model showing independent (fully adjusted) associations between selected baseline characteristics, including treatment assignment, and attainment or maintenance of A1C<6.5% or <7.0%.

Characteristics were selected by having unadjusted association with p<0.1. Attainment refers to the value at 1 year; maintenance refers to having a mean of yearly measurements including years 1 up to 5. OR=Odds Ratio; CI=Confidence Interval.

Appendix Table 1: Clinical characteristics of participants at enrollment, by randomized treatment groups (insulin glargine or standard therapy) and by subgroups according to glycemic status (diabetes or not diabetes). Values are given as percentage, mean (standard deviation), or median (inter-quartile range) as appropriate.

Appendix Table 2: Logistic regression model showing unadjusted associations between baseline characteristics and attainment or maintenance of A1C<6.5% or 7.0%. Attainment refers to the value at 1 year; maintenance refers to the mean of yearly measurements including years 1 up to 5. OR=Odds Ratio; CI=Confidence Interval.

Appendix Table 3: Glucose-lowering therapies used before randomized treatment (A) and at the end of treatment (B) by glycemic status at baseline and by treatment assignment.

Table 9: Logistic regression model showing independent (fully adjusted) associations between selected baseline characteristics, including treatment assignment, and attainment or maintenance of A1C<6.5% or <7.0%. Characteristics were selected by having unadjusted association with p<0.1. Attainment refers to the value at 1 year; maintenance refers to having a mean of yearly measurements including years 1 up to 5. OR=Odds Ratio; CI=Confidence Interval.

### Example 12

### Conclusions

Whether insulin therapy is beneficial, harmful or neutral with respect to cardiovascular outcomes has been debated for years. The ORIGIN trial was the first outcomes trial to explicitly test the cardiovascular effect of insulin. It showed that targeting and achieving normal or near-normal fasting glucose levels with basal insulin for a period of 5-7 years neither reduces nor increases serious cardiovascular outcomes compared to achieving guideline-suggested glucose levels without insulin. Thus, this intervention either has no cardiovascular effects or a longer period of observation is required to detect any effect. This latter possibility is supported by 2 different trials in patients with type 2 diabetes^{10, 18} and 1 trial in patients with type 1 diabetes¹⁹ in which a cardiovascular benefit that was not apparent at the end of the active treatment period of 6-10 years emerged after an additional 8-10 years of passive follow-up.

ORIGIN also showed that near-normal FPG and A1C levels can be achieved and maintained for more than 6 years by adding 1 injection of basal insulin to 0 or 1 oral agents when self-monitored fasting glucose levels are used by high-risk patients to self-titrate insulin glargine.

It is notable that the intervention reduced the incidence of new diabetes both using the protocol's definition of diabetes (i.e. based on new cases up to and including the results of the first oral glucose tolerance test done 1 month after insulin was stopped), and after all possible cases of diabetes were included in a sensitivity analysis. This may be due to some preservation of beta cell function in response to several years of a reduced need to secrete all of the required insulin, or a direct effect of insulin on the beta cell, and is unlikely to be due to any masking of hyperglycemia by exogenous glargine insulin as its mean duration of action is approximately 20 hours²⁰.

Participants allocated to insulin glargine experienced more hypoglycemia than standard care participants; however the absolute risk of severe and nonsevere hypoglycemia in this population was low (i.e. approximately 0.7 more severe episodes and 11 more suspected or confirmed episodes per 100 person-years). This and the observation that 43% of insulin glargine participants did not experience even 1 episode over a median of 6.2 years of followup may have been due to the inclusion of people with relatively recent dysglycemia; the use of insulin glargine with its long duration of action; the fact that basal and not prandial insulin was used; and the concomitant use of metformin in 47% of participants. Nevertheless the risk of hypoglycemia was approximately 3-fold higher than in standard care participants and insulin glargine participants experienced a 1.6 kg weight gain. The fact that no differences in cardiovascular outcomes were noted suggests that these adverse effects do not increase serious outcomes.

ORIGIN had several strengths. A clear and consistent difference in therapy was achieved and maintained between treatment groups. Thus, more than 50% of people allocated to insulin glargine titrated the dose sufficiently to achieve FPG levels below 5.3 mmol/l (95 mg/dl) and 75% of them achieved FPG levels ≦6.0 mmol/l (108 mg/dl) for most of the trial. This was in contrast to standard care participants who used oral agents to manage glycemia and achieved a final median FPG level of 6.8 mmol/l and A1C levels consistent with those recommended in clinical practice guidelines, and who used very little insulin throughout the trial. The trial duration of more than 6 years, the high follow-up rates in both groups, the large number of cardiovascular outcomes (2.9 and 5.4 per 100 person-years for the primary composite and co-primary expanded composite outcome respectively), and prospective collection and adjudication of these outcomes ensured that the study had sufficient power to detect a clinically important short or medium-term cardiovascular effect of the intervention. Finally, the prospective collection of data pertaining to nonsevere and severe hypoglycemia, weight gain and cancers ensured that harms were detected and quantified.

ORIGIN's findings should reassure clinicians and patients of the overall cardiovascular safety of basal insulin in general and insulin glargine in particular in people at high risk for cardiovascular outcomes with early dysglycemia. Specifically, it does not increase cardiovascular or other serious long-term health outcomes compared to non-insulin based approaches to glucose lowering despite more hypoglycemia. The fact that exogenous insulin did not increase cardiovascular outcomes in this population also alleviates concerns regarding the cardiovascular effect of providing insulin to individuals who are likely to be insulin resistant (such as those who participated in ORIGIN).

### LIST OF ABBREVIATIONS AND DEFINITIONS OF TERMS

ABI Ankle-Brachial Index
AE adverse event
AGI alpha glucosidase inhibitor
ALT alanine aminotransferase
anti-GAD Ab anti-glutamic acid decarboxylase antibody
AST aspartate aminotransferase
BG blood glucose
BID twice a day (bis in die)
BMI Body Mass Index
BP blood pressure
CABG coronary artery bypass grafting
CI confidence interval
CV cardiovascular
EAC Event Adjudication Committee
ED erectile dysfunction
EOS end-of-study
EUF end-of-usual-follow-up
FPG fasting plasma glucose
HbA1c glycosylated hemoglobin A1c
HDL high-density lipoprotein
HGM home glucose monitoring
HIV human immunodeficiency virus
ICU Intensive Care Unit
IEC Independent Ethics Committee
IFG impaired fasting glucose
IGT impaired glucose tolerance
ITT intention-to-treat
IV intravenous
LDL low-density lipoprotein
LVH left ventricular hypertrophy
MedDRA Medical Dictionary for Regulatory Affairs
MET metformin
MGT meglitinide
MI myocardial infarction
MSE Mental Status Exam
NGT normal glucose tolerance
n-IgI normalized insulinogenic index
NYHA New York Heart Association
OAD oral antidiabetic drug
OGTT oral glucose tolerance test
omega-3 omega-3 polyunsaturated fatty acids
PUFA
PCI percutaneous intervention
PO per os (orally)
PPG postprandial plasma glucose
PTCA percutaneous transluminal coronary angioplasty
QD once a day (quaque die)
SC subcutaneous
SD standard deviation
SU sulfonylurea
T1 DM type 1 diabetes mellitus
T2DM type 2 diabetes mellitus
TZD Thiazolidinedione
VLDL Very low density lipoprotein
vs versus
UKPDS United Kingdom Prospective Diabetes Study
ULN upper limit of normal
WESDR Wisconsin Epidemiologic Survey of Diabetic Rethinopathy

### REFERENCES

1. Sarwar N, Gao P, Seshasai S R et al. Diabetes mellitus, fasting blood glucose concentration, and risk of vascular disease: a collaborative meta-analysis of 102 prospective studies. Lancet 2010; 375(9733):2215-2222.
2. Selvin E, Steffes M W, Zhu H et al. Glycated hemoglobin, diabetes, and cardiovascular risk in nondiabetic adults. N Engl J Med 2010; 362(9):800-811.
3. Gerstein H C, Santaguida P, Raina P et al. Annual incidence and relative risk of diabetes in people with various categories of dysglycemia: A systematic overview and meta-analysis of prospective studies. Diabetes Res Clin Pract 2007; 78(3):305-312.
4. Anand S S, Dagenais G R, Mohan V et al. Glucose levels are associated with cardiovascular disease and death in an international cohort of normal glycaemic and dysglycaemic men and women: the EpiDREAM cohort study. Eur J Cardiovasc Prev Rehabil 2011.
5. Gerstein H C, Islam S, Anand S et al. Dysglycaemia and the risk of acute myocardial infarction in multiple ethnic groups: an analysis of 15,780 patients from the INTERHEART study. Diabetologia 2010.
6. Gerstein H C. More insights on the dysglycaemia-cardiovascular connection. Lancet 2010; 375(9733):2195-2196.
7. Seshasai S R, Kaptoge S, Thompson A et al. Diabetes mellitus, fasting glucose, and risk of cause-specific death. N Engl J Med 2011; 364(9):829-841.
8. Turnbull F M, Abraira C, Anderson R J et al. Intensive glucose control and macrovascular outcomes in type 2 diabetes. Diabetologia 2009; 52(11):2288-2298.
9. Action to Control Cardiovascular Risk in Diabetes Study Group., Gerstein H C, Miller M E et al. Effects of intensive glucose lowering in type 2 diabetes. N Engl J Med 2008; 358(24):2545-2559.
10. Holman R R, Paul S K, Bethel M A, Matthews D R, Neil H A. 10-Year Follow-up of Intensive Glucose Control in Type 2 Diabetes. N Engl J Med 2008; 359(15):1577-1589.
11. Vehkavaara S, Yki-Jarvinen H. 3.5 years of insulin therapy with insulin glargine improves in vivo endothelial function in type 2 diabetes. Arterioscler Thromb Vasc Biol 2004; 24(2):325-330.
12. Franklin V L, Khan F, Kennedy G, Belch J J, Greene S A. Intensive insulin therapy improves endothelial function and microvascular reactivity in young people with type 1 diabetes. Diabetologia 2008; 51(2):353-360.
13. Dandona P, Chaudhuri A, Ghanim H, Mohanty P. Proinflammatory effects of glucose and anti-inflammatory effect of insulin: relevance to cardiovascular disease. Am J Cardiol 2007; 99(4A):15B-26B.
14. Li Y, Xu W, Liao Z et al. Induction of long-term glycemic control in newly diagnosed type 2 diabetic patients is associated with improvement of beta-cell function. Diabetes Care 2004; 27(11):2597-2602.
15. Weng J, Li Y, Xu W et al. Effect of intensive insulin therapy on beta-cell function and glycaemic control in patients with newly diagnosed type 2 diabetes: a multicentre randomised parallel-group trial. Lancet 2008; 371(9626):1753-1760.
16. Hu Y, Li L, Xu Y et al. Short-term intensive therapy in newly diagnosed type 2 diabetes partially restores both insulin sensitivity and beta-cell function in subjects with long-term remission. Diabetes Care 2011; 34(8):1848-1853.
17. Origin Trial Investigators. Rationale, design, and baseline characteristics for a large international trial of cardiovascular disease prevention in people with dysglycemia: the ORIGIN Trial (Outcome Reduction with an Initial Glargine Intervention). Am Heart J 2008; 155(1):26-32.
18. Stamler J, Vaccaro O, Norton J D, Wentworth D. Diabetes, other risk factors, and 12-yr cardiovascular mortality for men screened in the Multiple Risk Factor Intervention Trial. Diabetes Care 1993; 16: 434-44.
19. The Diabetes Control and Complications Trial Research Group: the effect of intensive treatment of diabetes on the development and progression of long-term complications in insulin-dependent diabetes mellitus. N Engl J Med 1993; 329: 977-86.
20. Shichiri M, Kishikawa H, Ohkubo Y, Wake N. Long-term results of the Kumamoto study on optimal diabetes control in type 2 diabetic subjects. Diabetes Care 2000 April; 23(Supp2): B21-9.
21. Reichard P, Nilsson B-Y, Rosenqvist U. The effect of long-term intensified insulin treatment on the development of microvascular complications of diabetes mellitus. N Engl J Med 1993; 329: 304-9.
22. UKPDS Group. Intensive blood-glucose control with sulphonylureas or insulin compared with conventional treatment and risk of complications in subjects with type 2 diabetes (UKPDS 33). Lancet 1998; 352: 837-53.
23. Laakso M. Glycemic control and the risk for coronary heart disease in subjects with non-insulin-dependent diabetes mellitus. Annals Int Med 1996; 124(1 pt 2): 127-130.
24. Moss S E, Klein R, Klein B E K, Meuer S M. The association of glycemia and cause-specific mortality in a diabetic population. Arch Int Med 1994; 154: 2473-9.
25. Jackson C A, Yudkin J S, Forrest R D. A comparison of the relationships of the glucose tolerance test and the glycated haemogoblin assay with diabetic vascular disease in the community. The Islington Diabetes Survey. Diabetes Res Clin pract 1992; 17: 111-123.
26. Wei M, Gaskill S P, Haffner S M, Stern M P. Effects of diabetes and level of glycemia on all-cause and cardiovascular mortality, The San Antonio Heart Study. Diabetes Care 1198; 21(7): 1167-72.
27. Expert Committee on the Diagnosis and Classification of Diabetes Mellitus. Report of the expert committee on the diagnosis and classification of diabetes mellitus. Diabetes Care 1997; 20(7): 1183-97.
28. Stratton I M, Adler A I, Neil A W, Matthews D R, Manley S E, Cull C A, et al. Association of glycaemia with macrovascular and microvascular complications of type 2 diabetes (UKPDS 35): prospective observational study. BMJ 2000; 321: 405-412.
29. Coutinho M, Wang Y, Gerstein H C, Yusuf S. The relationship between glucose and incident cardiovascular events. Diabetes Care 1999; 22(2): 233-240.
30. Khaw K-T, Wareham N, Luben R, Bingham S, Oakes S, Welch A, et al. Glycated haemoglobin, diabetes, and mortality in men in the Norfolk cohort of European Prospective Investigation of Cancer and Nutrition (EPIC-Norfolk). BMJ 2001; 322: 15-18.
31. Gerstein H C, Yusuf S. Dysglycaemia and risk of cardiovascular disease. Lancet 1996; 347: 949-50.
32. deVegt F, Dekker J M, Ruhé H G, Stehouwer C D A, Nijpels G, Bouter L M, et al. Hyperglycaemia is associated with all-cause and cardiovascular mortality in the Hoorn population: the Hoorn Study. Diabetologia 1999; 42: 926-931.
33. Simons L A, McCallum J, Friedlander Y, Simons J. Fasting plasma glucose in non-diabetic elderly women predicts increased all-cause mortality and coronary heart disease risk. Aust N Z Med 2000; 30: 41-7.
34. Balkau B, Shipley M, Jarret R J, Pyorala K, Pyorala M, Forhan A. et al. High blood glucose concentration is a risk factor for mortality in middle-aged nondiabetic men. 20-year follow-up in the Whitehall Study, the Paris Prospective Study, and the Helsinki Policemen Study. Diabetes Care 1998; 21: 360-367.
35. Bjornholt J V, Nitter-Hauge S, Erikssen G, Jervell J, Aaser E, Erikssen J, et al. Fasting blood glucose: an underestimated risk factor for cardiovascular death. Diabetes Care 1999; 22: 45-9.
36. Balkau B, Bertrais S, Dugimetiere P, Eschwege E. Is there a glycemic threshold for mortality risk? Diabetes Care 1999; 22(5): 696-9.
37. Harper C R, Jacobson T A. The fats of life. The role of omega-3 fatty acids in the prevention of coronary artery disease. Arch Int Med 2001; 161:2185-92.
38. Burr M L, Fehily A M, Gilbert J F, et al. Effects of changes in fat, fish, and fiber intakes on death and myocardial infarction: diet and reinfarction trial (DART). Lancet 1989; ii:757-761.
39. De Longeril M, Renaud S, Mamelle N, et al. Mediterranean alpha-linolenic acid-rich diet in secondary prevention of coronary heart disease. Lancet 1994; 343:1454-1459.
40. GISSI Prevenzione Investigators. Dietary supplementation with n-3 polyunsaturated fatty acids and Vitamin E after myocardial infarction: results of the GISSI Prevenzione Trial. Lancet 1999; 354:447-455.
41. Connor W E. Fish oil in hypertriglyceridemia: safety and recommendations. Lipids 1999(suppl); 34:S271.
42. Malmberg K, Ryden L, Hamsten A, Herlitz J, Waldenstrom A, Wedel H. Mortality prediction in diabetic patients with myocardial infarction: experiences from the DIGAMI study. Cardiovascular Research 1997; 34: 248-253.
43. Van den Berghe G, Wouters P, Weekers F, Verwaest C, Bruyninckx F, Schetz M et al. Intensive insulin therapy in critically ill patients. N Engl J Med 2001; 345: 1359-67.
44. Opie L H. Metabolic response during impending myocardial infarction: relevance of studies of glucose and fatty acid metabolism in animals. Circulation 1972; 45: 483-90
45. Opie L H. Metabolism of free fatty acids, glucose, and catecholamines in acute myocardial infarction-relation to myocardial ischemia and infarct size. Am J Cardiol 1975; 20: 605-17
46. Oliver M F, Opie I H. Effects vof glucose and fatty acids in myocardial ischemia and arrhythmias Lancet 1994; 343: 155-8
47. Fath-Ordoubadi F, Beatt K J. Glucose-insulin-potassium therapy for treatment of acute myocardial infarction: an overview of randomized, placebo-controlled trials. Circulation 1997; 96: 1152-6
48. Rogers W J et al. Acute effects of glucose-insulin-potassium infusion on myocardial substrates, coronary blood flow, and oxygen consumption in man. Am J Cardiol 1977; 40: 421-8
49. Baron A D. Vascular reactivity. Am J Cardiol 1999; 84(1A): 25J-27J.
50. Aljada A, Dandona P. Effect of insulin on human aortic endothelial nitric oxide synthase. Metabolism 2000; 49: 147-50.
51. Taylor P D, Oon B B, Thomas C R, Poston T, Poston L. Prevention by insulin treatment of endothelial dysfunction but not enhanced noradrenaline-induced contractility in mesenteric resistance arteries from streptozotocin-induced diabetic rats. Br J Pharmacol 1994; 111(I): 35-41.
52. Dandona P, Aljada A, Mohanty P, Ghanim H, Hamouda W, Assian E, Ahmad S. Insulin inhibits intranuclear nuclear factor kB and stimulates IkB in mononuclear cells in obese subjects: evidence for an anti-flammatory effect? J Clin Endocrin; July 2001: 3257-3265.
53. Holman R R, Paul S K, Bethel M A, Matthews D R, Neil H A W. 10-year follow-up of intensive glucose control in type 2 diabetes. NEJM 2008; 359: 1577-89
54. The Action to Control CardiOvascular Risk in Diabetes Study Group. Effects of intensive glucose lowering in type 2 diabetes. NEJM 2008; 358: 2545-59.
55. The ADVANCE Collaborative Study Group. Intensive blood glucose control and vascular outcomes in patients with type 2 diabetes. NEJM 2008; 358: 2560-72.
56. Dormandy J A, Charbollel B, Eckland D J et al. Secondary prevention of macrovascular events in patients with type 2 diabetes in the PROactive Study (PROspective pioglitAzone Clinical Trial In macroVascular Events); a randomized clinical trial. Lancet 2005; 366: 1279-89
57. Data presented at American Diabetes Association Scientific Meetings, Jun. 8, 2008, San Francisco Calif.
58. The Diabetes Control and Complications Trial/Epidemiology of Diabetes Interventions and Complications (DCCT/EDIC) Study Research Group. Intensive diabetes treatment and cardiovascular disease in patients with type 1 diabetes. NEJM 2005; 353: 2643-53.
59. UKPDS Group. Effect of intensive blood glucose control with metformin on complications in overweight patients with type 2 diabetes (UKPDS 34). Lancet 1998; 352: 854-65.
60. ORIGIN Trial Investigators, GersteinH, Yusuf S, Riddle M C, Ryden L, Bosch J. Rationale, design, and baseline characteristics for a large international trial of cardiovascular disease prevention in people with dysglycemia: the ORIGIN trial. Am Heart J 2008: 155: 26-32.
61. Folstein M F, Folstein S E, McHugh P R (1975). ““Mini-mental state”. A practical method for grading the cognitive state of patients for the clinician”. Journal of psychiatric research 12 (3): 189-98

### REFERENCES FOR EXAMPLE 11

1. Stratton I M, Adler A I, Neil H A W, Matthews D R, Manley S E, Cull C A, Hadden D, Turner R C, Holman R R. Association of glycemia with macrovascular and microvascular complications of type 2 diabetes (UKPDS 35): prospective observational study. BMJ 2000; 321:405-12
2. Saydah S, Tao M, Imperatore G, Gregg E. GHb level and subsequent mortality among adults in the U.S. Diabetes Care 2009; 32:1440-1446
3. Sawar N, Gao P, Seshasai S R, Gobin R, Kaptoge S, DiAngelantonio E, Ingelsson E, Lawlor D A, Selvin E, Stampfer M, Stehouwer C D, Lewington S, Pennells L, Thompson A, Sattar N, White I R, Ray K K, Danesh J. Lancet 2010; 375:2215-2222
4. Gerstein H C, Islam S, Anand S, Almahmeed W, Damesceno A, Dans A, Lang C C, Luna M A, McQueen M, Rangarajan S, Rosengren A, Wang X, Yusuf S. Diabetologia 2010; 53:2509-2517
5. United Kingdom Prospective Study Group. Intensive blood-glucose control with sulphonylureas or insulin compared with conventional treatment and risk of complications in patients with type 2 diabetes (UKPDS 33). Lancet 1998; 352:837-853
6. United Kingdom Prospective Study Group. Effect of intensive blood-glucose control with metformin on complications in overweight patients with type 2 diabetes (UKPDS 34). Lancet 1998; 352:854-865
7. The ACCORD Study Group and ACCORD Eye Study Group. Effects of medical therapies on retinopathy progression in type 2 diabetes. N Engl J Med 2010; 363:233-244
8. Ismail-Beigi F, Craven T, Banerji M A, Basile J, Calles J, Cohen R M, Cuddihy R, Cushman W C, Genuth S, Grimm R H Jr, Hamilton B P, Hoogwerf B, Karl D, Katz L, Krikorian A, O'Connor P, Pop-Busui R, Schubart U, Simmons D, Taylor H, Thomas A, Weiss D, Hramiak I, for the ACCORD trial group. Lancet 2010; 376:419-430
9. Hadden D R, Blair A L T, Wilson E A, Boyle D Mc C, Atkinson A B, Kennedy A L, Buchanan K D, Merrett J D, Montgomery D A D, Weaver J A. Natural history of diabetes presenting age 40-69 Years: A prospective study of the influence of intensive dietary therapy. Quart J Med 1986; 2230:579-598
10. UK Prospective Diabetes Study Group. Overview of 6 years' therapy of type II diabetes: a progressive disease. 1995; 44:1249-1258
11. Kahn S E, Haffner S M, Heise M A, Herman W H, Holman R R, Jones N P, Kravitz B G, Lachlin J M, O'Neill C, Zinman B, Viberti G, for the ADOPT Study Group. Glycemic durability of rosiglitazone, metformin, or glyburide monotherapy. N Engl J Med 2006; 355:23:2427-2443
12. The ORIGIN Trial Investigators. Rationale, design, and baseline characteristics for a large international trial of cardiovascular disease prevention in people with dysglycemia: the ORIGIN Trial (Outcome Prevention with an Initial Glargine Interventin). Am Ht J 2008; 155:26-32.e6
13. The ORIGIN Trial Investigators. Basal insulin and cardiovascular and other outcomes in dysglycemia. N Engl J Med 2012; 367:319-328
14. The ORIGIN Trial Investigators. n-3 fatty acids and cardiovascular outcomes in patients with dysglycemia. N Engl J Med 2012; 367:308-318
15. American Diabetes Association. Standards of medical care in diabetes-2012. Diabetes Care 2012; 35(Suppl 1):S11-S63
16. Rodbard H W, Jellinger P S, Davidson H A, Einhorn D, Garber A J, Grunberger G, Handelsman Y, Horton E S, Lebovitz H, Levy P, Moghissi E S, Schwartz S S. Statement by an American Association of Clinical Endocrinologists/American College of Endocrinology consensus panel on type 2 diabetes mellitus: an algorithm for glycemic control. Endocrine Practice 2009; 15:541-559
17. The ADVANCE Collaborative Group. Intensive blood glucose control and vascular outcomes in patients with type 2 diabetes. N Engl J Med 2008; 358:2560-2572
18. The Action to Control Cardiovascular Risk in Diabetes Study Group. Effects of intensive glucose lowering in type 2 diabetes. N Engl J Med 2008; 358:2545-2559
19. Duckworth W, Abraira C, Moritz T, Reda D, Emanuele N, Reaven P D, Zieve F J, Marks J, Davis S N, Hayward R, Warren S R, Goldman S, McCarren M, Vitek M E, Henderson W G, Huang G D, for the VADT Investigators. Glucose control and vascular complications in veterans with type 2 diabetes. N Engl J Med 2009; 360:129-139
20. Brown J B, Nichols G A, Perry A. The burden of treatment failure in type 2 diabetes. Diabetes Care 2004; 27:1535-1540
21. Brown J B, Conner C, Nichols G A. Secondary failure of metformin monotherapy in clinical practice. Diabetes Care 2010; 33:501-506
22. Riddle M C, Yuen K C J. Revaluating goals of insulin therapy: perspectives from large clinical trials. Endocrinol Metab Clin NA 2012; 41:41-56
23. American Diabetes Association. Diagnosis and classification of diabetes mellitus. Diabetes Care 2012; 35(Supp1); 564-71
24. World Health Organization. Use of glycated haemoglobin (HbA1c) in the diagnosis of diabetes mellitus: abbreviated report of a WHO consultation. (http://www.who.int/diabetes/publications/diagnosis_diabetes2011/en/index.html)
25. Miller M E, Bonds D E, Gerstein H C, Seaquist E R, Bergenstal R M, Galles-Escandon J, Childress R D, Craven T E, Cuddihy R M, Gailey G, Feinglos M N, Ismail-Beigi F, Largay J F, O'Connor P J, Paul T, Savage P J, Schubart U K, Sood A, Genuth S, for the ACCORD investigators. BMJ 2010:340:b5444

TABLE 1
Baseline Characteristics

| | Insulin Glargine | Standard |
|---|---|---|
| | (N = 6264) | (N = 6273) |
| Mean (SD) Age (yrs) | 63.6 (7.8) | 63.5 (7.9) |
| N Females (%) | 2082 (33.2) | 2304 (36.7) |
| N Prior CV Event (%) | 3711 (59.3) | 3666 (58.4) |
| N Hypertension (%) | 4973 (79.5) | 4989 (79.5) |
| N Current Smoking (%) | 781 (12.5) | 771 (12.3) |
| N Any Albuminuria (%)* | 939 (15.0) | 985 (15.7) |
| N ABI ≦ 0.9 (%) | 470 (7.8) | 501 (8.3) |
| Glycemic Characteristics | | |
| N Prior DM on oral agent (%) | 3748 (59.8) | 3692 (58.9) |
| N Prior DM drug naive (%) | 1414 (22.6) | 1467 (23.4) |
| N New DM (%) | 365 (5.8) | 395 (6.3) |
| N IGT/IFG (%) | 735 (11.7) | 717 (11.4) |
| Mean (SD) Years of Diabetes | 5.5 (6.1) | 5.3 (5.9) |
| Median FPG (IQR) | 6.9 (6.1, 8.2) | 6.9 (6.0, 8.2) |
| Median A1c (IQR) | 6.4 (5.8, 7.2) | 6.4 (5.8, 7.2) |
| Glycemic Drugs | | |
| N Metformin (%) | 1694 (27.0) | 1741 (27.8) |
| N Sulfonylurea (%) | 1901 (30.3) | 1810 (28.9) |
| N Other (%) | 173 (2.7) | 178 (2.8) |
| N No Drugs (%) | 2501 (39.9) | 2551 (40.7) |
| Other CV Risk Factors | | |
| Mean (SD) Systolic BP (mm) | 146 (22) | 146 (22) |
| Mean (SD) Diastolic BP (mm) | 84 (12) | 84 (12) |
| Mean Weight (SD) | 83.3 (16.8) | 83.1 (17.3) |
| Mean (SD) Body Mass Index | 29.8 (5.2) | 29.9 (5.3) |
| Mean (SD) Waist/Hip Men | 0.99 (0.09) | 0.98 (0.09) |
| Mean (SD) Waist/Hip Women | 0.90 (0.09) | 0.90 (0.09) |
| Mean (SD) Cholesterol | 4.9 (1.2) | 4.9 (1.2) |
| Mean (SD) LDL Cholesterol | 2.9 (1.0) | 2.9 (1.0) |
| Mean (SD) HDL Cholesterol | 1.2 (0.3) | 1.2 (0.3) |
| Median (IQR) Triglyceride | 1.6 (1.1, 2.2) | 1.6 (1.1, 2.2) |
| Mean (SD) Creatinine | 89.2 (22.0) | 88.8 (22.1) |
| Mean eGFR | 77.5 (20.8) | 77.1 (21.8) |
| Median (IQR) Urine ACR* | 0.57 (0.27, 1.96) | 0.57 (0.27, 1.96) |
| Other Drugs | | |
| N Statin (%) | 3373 (53.9) | 3367 (53.7) |
| N ACE-I or ARB (%) | 4330 (69.2) | 4351 (69.4) |
| N Other BP Drug (%) | 4478 (71.5) | 4518 (72.0) |
| N Antiplatelet (%) | 4296 (68.6) | 4370 (69.7) |

TABLE 2
Insulin Use and Dose and Glycemic Indices during the Trial

| | Insulin Glargine Group | | | Standard Care Group | | | FPG (mM) | | A1c (%) | |
|---|---|---|---|---|---|---|---|---|---|---|
| | | On Glargine | Glargine Dose | | On Insulin | Insulin Dose | Insulin | Standard | Insulin | Standard |
| | N | N (%) | (U/kg)^{a} | N | N (%)^{b} | (U/kg) | Glargine | Care | Glargine | Care |
| Baseline | 6264 | 6264 | 0 | 6273 | 0 | 0 | 6.9 (6.1, 8.2) | 6.9 (6.0, 8.2) | 6.4 (5.8, 7.2) | 6.4 (5.8, 7.2) |
| Year 1 | 6107 | 5625 (92.1) | 0.28 (0.17, 0.42) | 6136 | 110 (1.8) | 0.20 (0.12, 0.35) | 5.2 (4.6, 5.9) | N/A | 5.9 (5.5, 6.4) | 6.2 (5.7, 6.9) |
| Year 2 | 5986 | 5392 (90.1) | 0.36 (0.23, 0.52) | 6003 | 208 (3.5) | 0.23 (0.14, 0.39) | 5.0 (4.4, 5.8) | 6.6 (5.7, 7.9) | 6.0 (5.5, 6.5) | 6.3 (5.8, 6.9) |
| Year 3 | 5818 | 5186 (89.1) | 0.38 (0.25, 0.55) | 5855 | 304 (5.2) | 0.23 (0.14, 0.40) | 5.0 (4.4, 5.7) | N/A | 6.0 (5.6, 6.6) | 6.4 (5.8, 7.0) |
| Year 4 | 5653 | 4949 (87.6) | 0.39 (0.26, 0.55) | 5689 | 399 (7.0) | 0.25 (0.14, 0.40) | 5.1 (4.5, 5.8) | N/A | 6.1 (5.7, 6.7) | 6.4 (5.9, 7.1) |
| Year 5 | 5493 | 4718 (85.9) | 0.39 (0.26, 0.57) | 5507 | 494 (9.0) | 0.26 (0.15, 0.43) | 5.1 (4.5, 6.0) | N/A | 6.2 (5.7, 6.8) | 6.5 (6.0, 7.2) |
| Year 6 | 3927 | 3281 (83.6) | 0.40 (0.27, 0.57) | 3924 | 392 (10.0) | 0.26 (0.15, 0.46) | 5.2 (4.6, 6.1) | N/A | 6.3 (5.8, 6.8) | 6.5 (6.0, 7.2) |
| Year 7 | 851 | 713 (83.8) | 0.41 (0.26, 0.59) | 860 | 99 (11.5) | 0.22 (0.14, 0.50) | 5.2 (4.5, 6.3) | N/A | 6.2 (5.8, 7.1) | 6.5 (6.0, 7.1) |

All measured values are expressed as medians (interquartile range);
^{a}weight at the time of the visit was used for calculations;
^{b}not glargine insulin

TABLE 3
Glycemic and Cardiovascular Drugs Used at Study End

| | | Glargine | Standard | |
|---|---|---|---|---|
| | N | N (%) | N (%) | P |
| Metformin¹ | 10529 | 2443 (46.5) | 3154 (59.8) | <0.001 |
| Sulfonylurea¹ | 10529 | 1292 (24.6) | 2454 (46.5) | <0.001 |
| No Oral Agents¹ | 10529 | 1844 (35.1) | 1009 (19.1) | <0.001 |
| 1 Oral Agents¹ | 10529 | 2661 (50.6) | 2034 (38.6) | <0.001 |
| 2 Oral Agents¹ | 10529 | 603 (11.5) | 1470 (27.9) | <0.001 |
| ≧3 Oral Agents¹ | 10529 | 146 (2.8) | 762 (14.4) | <0.001 |
| Statin | 10246 | 3180 (62.2) | 3097 (60.4) | 0.06 |
| ACE-I/ARB | 10270 | 3933 (76.7) | 3926 (76.4) | 0.71 |
| Antiplatelet | 10248 | 3645 (71.2) | 3629 (70.7) | 0.55 |

¹Oral agents refers to oral antidiabetic drugs being taken at the penultimate visit (i.e. before any insulin was changed or stopped)

TABLE 4
Episode of Hypoglycemia

| | Insulin | Standard | P |
|---|---|---|---|
| | Glargine | Care | |
| Severe Hypoglycemia | | | |
| ≧1 Episode (N/100py)^{a} | 359 (1.00) | 113 (0.31) | <0.001 |
| Total Episodes Durinng | 457 | 134 | |
| Follow-up | | | |
| Confirmed Symptomatic | | | |
| Hypoglycemia | | | |
| ≧1 Confirmed Episode | 2612 (9.81) | 904 (2.68) | <0.001 |
| (N/100py)^{b} | | | |
| Mean Number of Episodes/yr | 1.3 (2.3) | 0.8 (1.2) | <0.001 |
| No Confirmed Episodes During | 3652 (58.3) | 5369 (85.6) | <0.001 |
| follow-up (%) | | | |
| Any Symptomatic Hypoglycemia | | | |
| ≧1 Episode (N/100py)^{c} | 3573 (16.73) | 1579 (5.16) | <0.001 |
| Mean Number of Episodes/yr | 2.6 (4.1) | 1.3 (2.3) | <0.001 |
| No Episodes During | 2691 (43.0) | 4694 (74.8) | <0.001 |
| Follow-up (%) | | | |

py-person-years;
^{a}requiring assistance that was either confirmed by a self-measured or laboratory plasma glucose level ≦ 2 mmol/l (36 mg/dl) or that recovered promptly after oral carbohydrate, intravenous glucose, or glucagon administration;
^{b}any symptomatic nonsevere hypoglycemic episode that was confirmed by a self-measured glucose level ≦ 3 mmol/l (54 mg/dl);
^{c}any symptomatic nonsevere hypoglycemic episode for which there was no confirmatory glucose level.

TABLE 5
Glargine Adherence

| | Ever Off | Permanently Off |
|---|---|---|
| N Randomized | 6264 | 6264 |
| N stopped drug (%) | 2671 (42.6) | 1060 (16.9) |
| Reason Stopped | | |
| N Hypoglycemia (%) | 218 (8.2) | 42 (4.0) |
| N Wt Gain (%) | 4 (0.15) | 4 (0.4) |
| N Hyperglycemia (%) | 4 (0.15) | 2 (0.2) |
| N Refusal (%) | 1903 (71.3) | 884 (83.4) |
| N Other (%) | 542 (20.3) | 128 (12.1) |

Rows are mutually exclusive. The first time participants went off was used to count the reasons for “ever off”. The last time they were off and stayed off was used to count the reasons for “permanently off”.

TABLE 6
Microvascular outcomes

| | | | | | Standard | |
|---|---|---|---|---|---|---|
| | | | Glargine | | N | |
| | HR (95% CI) | P | N (%) | Rate | (%) | Rate |
| Microvascular | 0.90 (0.81, 1.01) | 0.066 | 1280 | 3.7 | 1327 | 3.9 |
| Clin Microvasc | 0.74 (0.56, 0.98) | 0.038 | 190 | 0.5 | 222 | 3.5 |
| Laser/Vitrect | 0.59 (0.38, 0.91) | 0.016 | 79 | 0.22 | 90 | 0.24 |
| Renal Failure | 1.27 (0.67, 2.39) | 0.46 | 36 | 0.1 | 45 | 0.7 |
| Double Cr | 0.70 (0.46, 1.06) | 0.09 | 94 | 0.25 | 110 | 0.3 |
| Albumin Prog | 0.92 (0.82, 1.04) | 0.17 | 1154 | 3.3 | 1173 | 3.4 |

TABLE 7
Cognition outcomes

| | Glargine | Standard | |
|---|---|---|---|
| | N (%) | N (%) | P |
| Baseline | 483 (7.7) | 520 (8.3) | 0.232 |
| 2 years | 433 (7.6) | 440 (7.6) | 0.823 |
| ~4 years | 449 (9.0) | 518 (10.3) | 0.022 |
| End of | 464 (9.6) | 492 (10.2) | 0.358 |
| study | | | |
| Any | 605 (10.4) | 666 (11.3) | 0.075 |
| measure | | | |

TABLE 8

| | Overall | | Glargine | | | | |
|---|---|---|---|---|---|---|---|
| | ALL | | No diabetes | | Diabetes | | P |
| Randomized | 12537 | | 737 | | 5527 | | |
| Age | 63 | (8) | 64 | (8) | 63.53 | (7.77) | 0.661 |
| Females | 4386 | (35%) | 206 | (27.95%) | 1876 | (33.94%) | 0.001 |
| Prior CV Event | 7378 | (58.85%) | 532 | (72.18%) | 3180 | (57.54%) | <0.001 |
| Hypertension | 9963 | (79.47%) | 555 | (75.31%) | 4419 | (79.95%) | 0.003 |
| Current Smoker | 1552 | (12.38%) | 101 | (13.70%) | 680 | (12.30%) | 0.280 |
| >2 drinks/week | 2848 | (22.72%) | 243 | (32.97%) | 1175 | (21.26%) | <0.001 |
| >12 years education | 4739 | (37.80%) | 271 | (36.77%) | 2111 | (38.19%) | 0.455 |
| Depression | 1134 | (9.05%) | 82 | (11.13%) | 481 | (8.70%) | 0.031 |
| ABI ≦0.9 | 971 | (7.75%) | 50 | (6.78%) | 420 | (7.60%) | 0.430 |
| FPG | 6.94 | (6.06-8.20) | 6.10 | (5.50-6.40) | 7.20 | (6.20-8.40) | <0.001 |
| 2 hr PG | 9.50 | (8.17-11.56) | 8.60 | (7.80-9.61) | 12.18 | (10.45-14.13) | <0.001 |
| A1c | 6.40 | (5.81-7.18) | 5.70 | (5.40-6.00) | 6.55 | (6.00-7.29) | <0.001 |
| SBP | 145.79 | (21.77) | 142.46 | (20.32) | 146.36 | (21.67) | <0.001 |
| DBP | 84.13 | (12.07) | 83.90 | (11.77) | 84.21 | (12.03) | 0.509 |
| Grip Strength | 32.97 | (13.29) | 35.01 | (14.07) | 33.11 | (13.47) | <0.001 |
| Weight (kg) | 83.24 | (17.03) | 84.98 | (15.30) | 83.12 | (16.94) | 0.002 |
| BMI kg/m2 | 29.83 | (5.25) | 29.88 | (4.85) | 29.75 | (5.21) | 0.516 |
| Waist/Hip - Male | 0.98 | (0.09) | 0.99 | (0.09) | 0.99 | (0.09) | 0.535 |
| Waist/Hip - Female | 0.90 | (0.09) | 0.89 | (0.08) | 0.90 | (0.09) | 0.055 |
| Urine ACR | 0.58 | (0.28-2.11) | 0.44 | (0.24-1.00) | 0.62 | (0.28-2.40) | <0.001 |
| eGFR | 77.30 | (21.29) | 75.19 | (17.43) | 77.76 | (21.19) | <0.001 |
| ALT | 23.58 | (13.24) | 24.22 | (14.19) | 23.49 | (13.02) | 0.205 |
| Total Cholesterol | 4.90 | (1.20) | 4.75 | (1.11) | 4.93 | (1.21) | <0.001 |
| LDL | 2.90 | (1.03) | 2.78 | (1.02) | 2.92 | (1.04) | <0.001 |
| HDL | 1.19 | (0.32) | 1.20 | (0.31) | 1.18 | (0.32) | 0.300 |
| TG | 1.58 | (1.10-2.20) | 1.50 | (1.10-2.20) | 1.60 | (1.12-2.22) | 0.207 |
| Statin | 6740 | (53.76%) | 527 | (71.51%) | 2846 | (51.49%) | <0.001 |
| Thiazide | 2371 | (18.91%) | 150 | (20.35%) | 997 | (18.04%) | 0.127 |
| Beta Blocker | 6598 | (52.63%) | 493 | (66.89%) | 2780 | (50.30%) | <0.001 |
| ACE-I/ARB | 8681 | (69.24%) | 505 | (68.52%) | 3825 | (69.21%) | 0.705 |
| Omega-3 FA | 6281 | (50.10%) | 371 | (50.34%) | 2753 | (49.81%) | 0.787 |

| | Standard therapy | | | | |
|---|---|---|---|---|---|
| | No diabetes | | Diabetes | | P |
| Randomized | 719 | | 5554 | | <0.001 |
| Age | 63.77 | (7.95) | 63.51 | (7.84) | 0.406 |
| Females | 240 | (33.38%) | 2064 | (37.16%) | 0.048 |
| Prior CV Event | 510 | (70.93%) | 3156 | (56.82%) | <0.001 |
| Hypertension | 528 | (73.44%) | 4461 | (80.32%) | <0.001 |
| Current Smoker | 84 | (11.68%) | 687 | (12.37%) | 0.598 |
| >2 drinks/week | 216 | (30.04%) | 1214 | (21.86%) | <0.001 |
| >12 years education | 276 | (38.39%) | 2081 | (37.47%) | 0.632 |
| Depression | 89 | (12.38%) | 482 | (8.68%) | 0.001 |
| ABI ≦0.9 | 37 | (5.15%) | 464 | (8.35%) | 0.003 |
| FPG | 6.10 | (5.42-6.40) | 7.17 | (6.20-8.40) | <0.001 |
| 2 hr PG | 8.70 | (7.90-9.80) | 12.20 | (10.50-14.30) | <0.001 |
| A1c | 5.71 | (5.38-6.10) | 6.51 | (5.95-7.28) | <0.001 |
| SBP | 142.56 | (21.31) | 146.08 | (22.04) | <0.001 |
| DBP | 83.15 | (11.71) | 84.20 | (12.19) | 0.024 |
| Grip Strength | 33.27 | (13.12) | 32.53 | (13.00) | 0.156 |
| Weight (kg) | 83.89 | (16.53) | 83.04 | (17.38) | 0.197 |
| BMI kg/m2 | 29.60 | (5.10) | 29.92 | (5.36) | 0.118 |
| Waist/Hip - Male | 0.97 | (0.07) | 0.99 | (0.09) | 0.002 |
| Waist/Hip - Female | 0.89 | (0.09) | 0.90 | (0.10) | 0.105 |
| Urine ACR | 0.43 | (0.22-0.86) | 0.61 | (0.28-2.37) | <0.001 |
| eGFR | 75.83 | (18.23) | 77.31 | (22.19) | 0.046 |
| ALT | 24.23 | (13.30) | 23.51 | (13.32) | 0.188 |
| Total Cholesterol | 4.70 | (1.12) | 4.92 | (1.21) | <0.001 |
| LDL | 2.70 | (1.00) | 2.92 | (1.03) | <0.001 |
| HDL | 1.22 | (0.33) | 1.19 | (0.32) | 0.047 |
| TG | 1.50 | (1.02-2.10) | 1.60 | (1.12-2.22) | <0.001 |
| Statin | 500 | (69.54%) | 2867 | (51.62%) | <0.001 |
| Thiazide | 137 | (19.05%) | 1087 | (19.57%) | 0.742 |
| Beta Blocker | 485 | (67.45%) | 2840 | (51.13%) | <0.001 |
| ACE-I/ARB | 479 | (66.62%) | 3872 | (69.72%) | 0.090 |
| Omega-3 FA | 343 | (47.71%) | 2814 | (50.67%) | 0.135 |

TABLE 9

| | 1-year A1C | | | | 5-year mean A1C | | | |
|---|---|---|---|---|---|---|---|---|
| | 6.5% | | 7.0% | | 6.5% | | 7.0% | |
| | OR (95% CI) | p | OR (95% CI) | p | OR (95% CI) | p | OR (95% CI) | p |
| Age (per years) | | | 1.008 (1.001-1.016) | 0.025** | 1.014 (1.007-1.021) | <0.001** | 1.033 (1.024-1.041) | <0.001** |
| Females | | | | | | | 1.321 (1.120-1.557) | <0.001** |
| Prior CV Event | 0.88 (0.80-0.96) | 0.004** | 0.87 (0.78-0.97) | 0.014** | | | 0.863 (0.758-0.982) | 0.026** |
| Current | 0.866 (0.758-0.990) | 0.035** | | | | | | |
| Smoking | | | | | | | | |
| Alcohol >2/wk | 1.485 (1.327-1.661) | <0.001** | 1.516 (1.307-1.758) | <0.001** | 1.612 (1.414-1.838) | <0.001** | 1.864 (1.573-2.209) | <0.001** |
| Depression | | | | | 1.428 (1.180-1.727) | <0.001** | | |
| A1c (per %) | 0.340 (0.321-0.359) | <0.001** | 0.340 (0.319-0.363) | <0.001** | 0.193 (0.179-0.208) | <0.001** | 0.226 (0.209-0.245) | <0.001** |
| Waist/Hip | | | | | 0.273 (0.159-0.467) | <0.001** | | |
| (per unit) | | | | | | | | |
| Grip Strength | | | 1.008 (1.003-1.012) | <0.001** | 1.005 (1.000-1.009) | 0.038** | 1.014 (1.007-1.020) | <0.001** |
| (per kg) | | | | | | | | |
| Urine ACR | | | | | 0.998 (0.995-1.000) | 0.035** | 0.996 (0.994-0.999) | 0.003** |
| (per mg/g) | | | | | | | | |
| On ACE/ARB | | | | | | | 0.805 (0.704-0.921) | 0.002** |
| Diabetes | 0.415 (0.337-0.513) | <0.001** | 0.231 (0.153-0.347) | <0.001** | 0.309 (0.240-0.399) | <0.001** | 0.243 (0.151-0.391) | <0.001** |
| Glargine | 2.50 (2.29-2.74) | <0.001** | 2.735 (2.440-3.067) | <0.001** | 2.975 (2.671-3.315) | <0.001** | 2.408 (2.122-2.732) | <0.001** |

APPENDIX TABLE 1

| | 1 year A1C | | | | 5-year mean A1C | | | |
|---|---|---|---|---|---|---|---|---|
| | 6.5 | | 7.0 | | 6.5 | | 7.0 | |
| | OR (95% CI) | p | OR (95% CI) | p | OR (95% CI) | p | OR (95% CI) | p |
| Age (per years) | | | 1.008 (1.000-1.016) | 0.044** | 1.014 (1.007-1.022) | <0.001** | 1.031 (1.022-1.040) | <0.001** |
| Females | 0.952 (0.840-1.079) | 0.446 | 1.085 (0.930-1.266) | 0.301 | 1.036 (0.894-1.199) | 0.641 | 1.250 (1.051-1.486) | 0.012** |
| Prior CV Event | 0.868 (0.783-0.961) | 0.007** | 0.869 (0.766-0.986) | 0.029** | 0.933 (0.832-1.046) | 0.236 | 0.846 (0.738-0.971) | 0.017** |
| Current | 0.849 (0.739-0.975) | 0.020** | 0.910 (0.767-1.080) | 0.281 | 0.877 (0.746-1.032) | 0.114 | 0.922 (0.763-1.113) | 0.398 |
| Smoking | | | | | | | | |
| Education | 1.032 (0.937-1.138) | 0.522 | 1.124 (0.996-1.269) | 0.059 | 1.026 (0.921-1.143) | 0.643 | 1.087 (0.954-1.237) | 0.211 |
| >12 y | | | | | | | | |
| Alcohol >2/wk | 1.416 (1.255-1.598) | <0.001** | 1.481 (1.266-1.732) | <0.001** | 1.585 (1.391-1.807) | <0.001** | 1.815 (1.532-2.150) | <0.001** |
| Depression | 1.056 (0.898-1.242) | 0.511 | | | 1.439 (1.193-1.736) | <0.001** | 1.145 (0.911-1.438) | 0.246 |
| FPG | 1.008 (0.982-1.034) | 0.546 | 0.988 (0.959-1.017) | 0.419 | 0.993 (0.963-1.023) | 0.631 | 0.983 (0.952-1.015) | 0.300 |
| (per mmol/L) | | | | | | | | |
| A1c (per %) | 0.351 (0.330-0.374) | <0.001** | 0.357 (0.332-0.384) | <0.001** | 0.213 (0.197-0.230) | <0.001** | 0.242 (0.222-0.263) | <0.001** |
| SBP (per mm) | | | 1.000 (0.997-1.003) | 0.963 | 1.000 (0.998-1.003) | 0.993 | 1.001 (0.998-1.004) | 0.469 |
| Waist/Hip | | | | | 0.323 (0.185-0.562) | <0.001** | 0.693 (0.358-1.342) | 0.277 |
| (per unit) | | | | | | | | |
| Grip Strength | 1.004 (1.000-1.009) | 0.056 | 1.010 (1.004-1.016) | 0.001** | 1.008 (1.003-1.013) | 0.003** | 1.014 (1.007-1.020) | <0.001** |
| (per kg) | | | | | | | | |
| Urine ACR | 1.000 (0.998-1.001) | 0.575 | 0.999 (0.997-1.001) | 0.176 | 0.997 (0.995-1.000) | 0.023** | 0.996 (0.994-0.999) | 0.002** |
| (per mg/g) | | | | | | | | |
| ALT (per U/l) | 1.001 (0.997-1.005) | 0.528 | 1.003 (0.999-1.008) | 0.175 | | | | |
| On Stalin | 0.966 (0.874-1.068) | 0.499 | 1.020 (0.903-1.153) | 0.747 | 0.895 (0.800-1.001) | 0.053 | 1.080 (0.945-1.235) | 0.259 |
| On Thiazide | | | 1.110 (0.956-1.289) | 0.172 | 1.060 (0.928-1.210) | 0.391 | 1.021 (0.867-1.201) | 0.806 |
| On ACE/ARB | | | | | | | 0.785 (0.685-0.899) | <0.001** |
| Diabetes | 0.425 (0.342-0.529) | <0.001** | 0.253 (0.167-0.384) | <0.001** | 0.314 (0.244-0.404) | <0.001** | 0.228 (0.139-0.375) | <0.001** |

APPENDIX TABLE 2
Glucose-lowering therapies at baseline

| | No diabetes | | | Diabetes | | |
|---|---|---|---|---|---|---|
| | | Standard | | | Standard | |
| | Glargine | therapy | | Glargine | therapy | |
| | N (%) | N (%) | P | N (%) | N (%) | P |
| Randomized | 737 | 719 | | 5527 | 5554 | |
| No oral agents | 725 (98) | 705 (98) | 0.646 | 1776 (32) | 1846 (33) | 0.215 |
| Metformin | 8 (1.1) | 11 (1.5) | 0.455 | 1686 (30) | 1730 (31) | 0.463 |
| Sulfonylurea | 0 (0.00) | 2 (0.28) | 0.152 | 1901 (34) | 1808 (33) | 0.040 |
| Other oral agents | 4 (0.54) | 1 (0.14) | 0.188 | 169 (3.1) | 177 (3.2) | 0.696 |

Oral glucose-lowering therapies used prior to enrollment by glycemic status and treatment allocation. P-values for differences by treatment allocation are shown.

APPENDIX TABLE 3
Glucose-lowering therapies at end of treatment

| | No diabetes | | | | | Diabetes | | | | |
|---|---|---|---|---|---|---|---|---|---|---|
| | | | Standard | | | | | Standard | | |
| | Glargine | | therapy | | | Glargine | | therapy | | |
| | N (%) | | N (%) | | P | N (%) | | N (%) | | P |
| Randomized | 737 | | 719 | | | 5527 | | 5554 | | |
| No Oral Agents | 511 | (9.2) | 452 | (1.1) | <0.001 | 1343 | (29) | 564 | (12) | <0.001 |
| Metformin | 113 | (7.5) | 154 | (24) | 0.003 | 2341 | (50) | 3003 | (65) | <0.001 |
| Sulfonylurea | 21 | (3.3) | 47 | (7.4) | <0.001 | 1281 | (28) | 2410 | (52) | <0.001 |
| Metformin Alone | 37 | (5.7) | 128 | (20) | <0.001 | 290 | (6.3) | 1163 | (25) | <0.001 |
| Sulfonylurea Alone | 5 | (0.8) | 22 | (3.5) | <0.001 | 166 | (3.6) | 698 | (15) | <0.001 |
| Metformin + Sulfonylurea | 11 | (1.7) | 21 | (3.3) | 0.067 | 464 | (10) | 1540 | (33) | <0.001 |
| Metformin + Any other | 76 | (12) | 26 | (4.1) | <0.001 | 2051 | (44) | 1840 | (40) | <0.001 |
| Other OAD | 6 | (0.9) | 12 | (1.9) | 0.146 | 191 | (4.1) | 606 | (13) | <0.001 |
| Rapid or Regular Insulin | 4 | (0.6) | 2 | (0.3) | 0.423 | 99 | (2.1) | 263 | (5.7) | <0.001 |
| Any Insulin | 444 | (69) | 2 | (0.3) | <0.001 | 3801 | (82) | 570 | (12) | <0.001 |

Glucose-lowering therapies used at the end of treatment by glycemic status and treatment allocation. P-values for differences by treatment allocation are shown.

1. A method of reducing the risk of progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin reduces the risk of progression to type 2 diabetes in said patient.

2. A method of reducing the risk of a new angina in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces the risk of a new angina.

3. A method of reducing the risk of a microvascular event in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces the risk of a microvascular event.

4. A method for preventing the progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin reduces the risk of progression to type 2 diabetes in said patient.

5. A method for preventing a new angina in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces the risk of a new angina.

6. A method for preventing a microvascular event in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin prevents a microvascular event.

7. A method delaying the progression to type 2 diabetes in a patient diagnosed with a disease or condition selected from the group consisting of impaired fasting glucose (IFG) and impaired glucose tolerance (IGT), comprising administering to said patient a therapeutically effective dosage of a long acting insulin, wherein said therapeutically effective dosage of said long acting insulin delays the progression to type 2 diabetes in said patient.

8. A method according to claim 3, wherein the microvascular event is a clinical microvascular event.

9. A method according to claim 8, wherein the microvascular event is selected from a group comprising neuropathy, retinopathy and nephropathy.

10. A method according to claim 9, wherein the nephropathy is characterized by renal failure, end-stage renal disease, or renal death.

11. A method for reducing the risk for requiring treatment by laser surgery or vitrectomy in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting reduces the risk for requiring treatment by laser surgery or vitrectomy in said patient.

12. A method for reducing doubling of baseline serum creatinine in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces doubling of baseline serum creatinine in said patient.

13. A method for reducing the risk of cognitive impairment in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin reduces the risk of cognitive impairment in said patient.

14. A method according to claim 13, wherein the patient scores 24 or less in the Mini-Mental Status Exam (MMSE).

15. A method for lowering the triglyceride concentration in the blood in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin lowers the triglyceride concentration in the blood in said patient.

16. A method for lowering the cholesterol concentration in the blood in a patient diagnosed with a disease or condition selected from the group consisting of impaired fasting glucose (IFG), impaired glucose tolerance (IGT), and type 2 diabetes, wherein the patient diagnosed with type 2 diabetes is either drug naïve or receives an oral antidiabetic agent, comprising administering to said patient a therapeutically effective dosage of a long acting insulin, wherein said therapeutically effective dosage of said long acting insulin lowers the cholesterol concentration in the blood in said patient.

17. A method according to claim 3, wherein the patient has a HbA1c≧6.4 prior to administering the long-acting insulin.

18. A method according to claim 3, wherein the patient had a history of atrial fibrillation prior to administering the long-acting insulin.

19. A method according to claim 18, wherein the microvascular outcome is a clinical microvascular outcome.

20. A method according to claim 18, wherein the microvascular outcome is a laboratory-based microvascular outcome.

21. A method according to claim 18, wherein the microvascular outcome is a composite of: laser surgery or vitrectomy or blindness for diabetic retinopathy; development of renal death or the need for renal replacement treatment (dialysis or transplantation); doubling of serum creatinine; or progression from lesser to greater severity of microalbuminuria.

22. A method according to claim 3, wherein the long-acting insulin is selected from a group comprising insulin glargine, insulin detemir and insulin degludec.

23. A method according to claim 22, wherein the long-acting insulin is insulin glargine.

24. An article of manufacture comprising
a packaging material;
a long-acting insulin; and
a label or package insert contained within the packaging material indicating that patients receiving the treatment with the long-acting insulin can be treated by a method according to claim 3.

25. An article of manufacture comprising
a packaging material;
insulin glargine; and
a label or package insert contained within the packaging material indicating that patients receiving the treatment with the long-acting insulin can be treated by a method according to claim 3, wherein in such treatment the risk for cardiovascular outcomes, all-cause mortality or cancer is not altered when compared to standard glucose lowering therapy.

26. An article of manufacture according to claim 25, wherein the risk for cancer is not altered when compared to standard glucose lowering therapy with regard to any organ-specific type of cancer.

27. An article of manufacture according to claim 24, wherein the long-acting insulin is selected from a group comprising insulin glargine, insulin detemir and insulin degludec.

28. An article of manufacture according to claim 27, wherein the long-acting insulin is insulin glargine.
